(19) United States
(12) Patent Application Publication
(10) Pub. No.: US 2026/0216097 A1
(43) Pub. Date: Jul. 30, 2026

(54) METHODS AND COMPOSITIONS FOR TREATING EQUINE ASTHMA SYNDROME

(71) Applicant: Wiser Concepts, LLC, Versailles, KY (US)

(72) Inventor: Delia Nash, Versailles, KY (US)

(21) Appl. No.: 19/454,493

(22) Filed: Jan. 21, 2026

Related U.S. Application Data
(60) Provisional application No. 63/749,160, filed on Jan. 24, 2025.

(51) Int. Cl. A61K 31/05; A61K 9/00; A61K 31/198; A61K 31/352; A61K 31/7048; A61K 47/46; A61P 11/06

(52) U.S. Cl. CPC A61K 31/05; A61K 9/0053; A61K 31/198; A61K 31/352; A61K 31/7048; A61K 47/46; A61P 11/06

(57) ABSTRACT

Compositions for treating and/or preventing airway inflammation diseases or medical conditions in animals, such as Equine Asthma Syndrome, and methods of administering the compositions, are provided. The composition may be a feed supplement included in the diet of the animal. The composition may include one or more polyphenols, such as resveratrol, citrus bioflavonoid, hesperidin, and/or quercetin. In some embodiments, the composition may also include one or more amino acids, a carrier, and/or an additive.

### CROSS-REFERENCE TO RELATED APPLICATION

This application claims priority to U.S. Provisional Application No. 63/749,160, filed Jan. 24, 2025, the entire disclosure of which is hereby incorporated herein by reference.

### TECHNICAL FIELD

The present disclosure relates generally to nutritional compositions and methods of treatment and/or prevention of airway inflammatory diseases and medical conditions, particularly to those airway inflammation diseases and medical conditions that afflict animals such as equines.

### BACKGROUND

Equine asthma is a syndrome of non-infectious lower airway inflammation that commonly affects horses of all ages around the world. There has recently been a reclassification, grouping horses with mild lower airway inflammation, previously termed Inflammatory Airway Disease (IAD), and those with marked lower airway inflammation, previously known as Recurrent Airway Obstruction (RAO), into a unified syndrome, Equine Asthma Syndrome (EAS). Equine Asthma Syndrome represents this cohort of horses with lower airway inflammation that exhibit varying clinical presentations.

Treatment of horses afflicted by EAS is multifactorial. The mainstay of therapy varies based on severity and includes inhaled or systemic corticosteroids and bronchodilators, e.g., beta adrenergic agonists. Another vital part of management of EAS includes reduced exposure to respirable particles encountered in the normal stable environments, including dust, mold, and bacterial components, that typically trigger airway inflammation and clinical signs. This environmental modification, while vital, can be difficult to achieve and is potentially the greatest source of client non-compliance.

There exists a need for compositions and methods to treat horses afflicted by EAS and otherwise promote health in horses.

### SUMMARY

The present disclosure provides nutritional supplements and compositions for administration to a subject, such as a horse. These compositions and supplements may find use, for example, in treating and/or preventing Equine Asthma Syndrome (EAS).

For example, in one aspect, provided herein is a composition comprising one or more polyphenols, wherein the one or more polyphenols are present in a therapeutically effective amount for treating Equine Asthma Syndrome in an equine in need thereof, and wherein the one or more polyphenols comprise resveratrol and at least one of citrus bioflavonoid and hesperidin.

In another aspect, provided herein is a composition for the treatment of Equine Asthma Syndrome in an equine in need thereof. The composition may comprise resveratrol in an amount between 0.5% by weight to 10% by weight of the composition as a whole; and at least one of: citrus bioflavonoid in an amount between 5% by weight to 30% by weight of the composition as a whole; and hesperidin in an amount between 0.5% by weight to 10% by weight of the composition as a whole.

In another aspect, provided herein is a method for treating Equine Asthma Syndrome in an equine in need thereof. The method may comprise administering to the equine a composition comprising one or more polyphenols, wherein the one or more polyphenols are present in a therapeutically effective amount for treating Equine Asthma Syndrome in the equine, and wherein the one or more polyphenols comprise resveratrol and at least one of citrus bioflavonoid and hesperidin.

In another aspect, provided herein is a method for treating Equine Asthma Syndrome in an equine in need thereof. The method may comprise administering to the equine a composition comprising resveratrol in an amount between 0.5% by weight to 10% by weight of the composition as a whole; and at least one of: citrus bioflavonoid in an amount between 5% by weight to 30% by weight of the composition as a whole; and hesperidin in an amount between 0.5% by weight to 10% by weight of the composition as a whole.

In another aspect, provided herein is a method for treating Equine Asthma Syndrome in an equine in need thereof. The method may comprise administering a composition including a therapeutically effective amount of one or more phenols to the animal.

In another aspect, provided herein is a method for treating an airway inflammation disease in an animal in need thereof. The method may comprise administering a composition including a therapeutically effective amount of one or more phenols to the animal.

In another aspect, provided herein is a method for treating airway inflammatory responses to environmental triggers in an animal in need thereof. The method may comprise administering a composition including a therapeutically effective amount of one or more phenols to the animal.

The following description and the appended figures set forth certain features for purposes of illustration. Advantages will become more apparent when reading the present disclosure in its entirety.

### BRIEF DESCRIPTION OF DRAWINGS

So that the manner where the above recited features may be understood in detail, a more particular description, briefly summarized above, may be had by reference to example aspects, some of which are illustrated in the appended drawings.

FIG. 1 shows bar graphs of respiratory rates (A) and the proportion of horses with abnormal rebreathing exam (B) before and after 6-8 weeks of mixed polyphenol supplementation in eight healthy horses (open circles) and ten horses previously diagnosed with mild equine asthma (closed circles).

FIG. 2 shows bar graphs of bronchoalveolar lavage (BAL) cytology findings before and after 6-8 weeks of mixed polyphenol supplementation in eight healthy horses (open circles and open triangles) and ten horses previously diagnosed with mild equine asthma (closed circles and closed triangles), where the absolute % of neutrophils (A) or eosinophils (B) in BAL fluid obtained before and after the supplement treatment period and the change (delta, before—after treatment) in BAL % neutrophils (A) or eosinophils (B) during this period are shown, and * depict significant differences (P<0.05) between groups or within groups over time.

FIG. 3 shows bar graphs of concentrations of interleukin (IL)-6 (A), IL-10 (B), IL-17(C), and tumor necrosis factor (TNF)-α (D) in bronchoalveolar lavage (BAL) fluid obtained before and after 6-8 weeks of mixed polyphenol supplementation in eight healthy horses (open circles and open triangles) and ten horses previously diagnosed with mild equine asthma (closed circles and closed triangles), where the absolute BAL cytokine concentrations before and after the supplement treatment period and the change (delta, before—after treatment) in serum cytokine concentrations over the study period are shown in both groups, and * depict significant differences (P<0.05) between groups or within groups over time.

FIG. 4 shows bar graphs of respiratory rates (A) and the proportion of horses with abnormal rebreathing exam (B) before (white bars) and after (grey bars) asthma induction with four weeks of dusty hay feeding in 10 horses with previously diagnosed mild equine asthma, where five horses were randomly assigned to be treated with a mixed polyphenol supplement for four weeks prior to and during the asthma induction study (black circles), while five horses were randomized into an untreated control group (open circles), the change in respiratory rate during the four-week asthma induction study (delta, before - after) is shown in the control group with open triangles and the treatment group with black triangles, and * depict significant differences in respiratory rate between groups at the denoted time-point.

FIG. 5 shows bar graphs of bronchoalveolar lavage (BAL) cytology findings before (white bars) and after (grey bars) asthma induction with four weeks of dusty hay feeding in 10 horses with previously diagnosed mild equine asthma, where five horses were randomly assigned to be treated with a mixed polyphenol supplement for four weeks prior to and during the asthma induction study (black circles, black triangles), while five horses were randomized into an untreated control group (open circles, open triangles), the absolute % of neutrophils (A) or eosinophils (B) in BAL fluid obtained before and after the asthma induction period and the change (delta, before—after treatment) in BAL % neutrophils (A) or eosinophils (B) during this period are shown, and * depict significant differences (P<0.05) within groups over time.

FIG. 6 shows bar graphs of serum concentrations of interleukin (IL)-6 (A), IL-10 (B), IL-17 (C), and tumor necrosis factor (TNF)-α (D) before and after 6-8 weeks of mixed polyphenol supplementation in eight healthy horses (open circles and open triangles) and ten horses previously diagnosed with mild equine asthma (closed circles and closed triangles), where the absolute serum cytokine concentrations before and after the supplement treatment period and the change (delta, before—after treatment) in serum cytokine concentrations over the study period are shown.

FIG. 7 shows bar graphs of concentrations of interleukin (IL)-6 (A), IL-10 (B), and tumor necrosis factor (TNF)-α (C) in bronchoalveolar lavage (BAL) fluid obtained before and after asthma induction with four weeks of dusty hay feeding in 10 horses with previously diagnosed mild equine asthma, where five horses were randomly assigned to be treated with a mixed polyphenol supplement for four weeks prior to and during the asthma induction study (black circles, black triangles), while five horses were randomized into an untreated control group (open circles, open triangles), and the absolute serum cytokine concentrations before and after the supplement treatment period and the change (delta, before—after treatment) in BAL cytokine concentrations over the study period are shown.

### DETAILED DESCRIPTION

One or more specific embodiments of the present disclosure will be described herein. These described embodiments are only examples of the presently disclosed techniques. Additionally, in an effort to provide a concise description of these embodiments, all features of an actual implementation may not be described in the specification. It should be appreciated that in the development of any such actual implementation, as in any engineering or design project, numerous implementation-specific decisions must be made to achieve the developers'specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

### DEFINITIONS

The singular forms “a”, “an” and “the” include the plural forms as well, unless expressly stated otherwise or the context clearly indicates otherwise. For example, when introducing elements of various embodiments of the present disclosure, the articles “a,” “an,” and “the” are intended to mean that there are one or more of the elements.

The terms “comprising,” “including,” and “having” are intended to be inclusive and mean that there may be additional elements other than the listed elements. The term “comprising” may be considered synonymous with the term “including” for purposes of United States law. Whenever a composition, an element or a group of elements described herein is preceded with the transitional phrase “comprising” or “including”, and derivatives and equivalents thereof, it is understood that, in some embodiments, the transitional phrases “consisting essentially of,” “consisting of,” “selected from the group of consisting of,” or “is” precede the recitation of the composition, element, or elements and vice versa.

References to “one embodiment” or “an embodiment” of the present disclosure do not exclude the existence of additional embodiments that also incorporate the recited features.

The terms “first”, “second”, and the like are used herein to describe various features or elements, but these features or elements are not limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present disclosure.

The phrases, unless otherwise specified, “consists essentially of” and “consisting essentially of” do not exclude the presence of other steps, elements, or materials, whether or not, specifically mentioned in this specification, so long as such steps, elements, or materials, do not affect the basic and novel characteristics of the present disclosure, additionally, they do not exclude impurities and variances normally associated with the elements and materials used.

The terms “about” and “approximately” shall generally mean an acceptable degree of error or variation for the quantity measured given the nature or precision of the measurements, including experimental error and variations that would be expected by a person having ordinary skill in the art. Typical exemplary degrees of error or variation are within 20 percent (%), preferably within 10%, and more preferably within 5% of a given value or range of values. For biological systems, the term “about” refers to an acceptable standard deviation of error, preferably not more than 2-fold of a given value. Numerical quantities given herein are approximate unless stated otherwise, meaning that the term “about” or “approximately” can be inferred when not expressly stated.

Numerical ranges used herein include the numbers recited in the range unless otherwise specified. For example, unless otherwise specified, the numerical range “from/between 1% by weight to 10% by weight” or “from/between 1% by weight and 10% by weight” includes 1% by weight and 10% by weight within the recited range. For the sake of brevity, only some ranges may be explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

As used herein, “substantially free of” means that only trace components of a particular component may be found in the composition. In an embodiment, “substantially free of” means less than 0.1 % by weight of the nutritional composition. In some embodiments, substantially free of means less than 0.01 % by weight of the nutritional composition. In other embodiments, substantially free of means less than 0.001% by weight of the nutritional composition. Amounts of the components in the nutritional composition can be expressed using “wt %” which means “% by weight of the nutritional composition” unless expressly stated otherwise.

The terms “prevention,” “prevent,” “preventing,” “suppression,” “suppress” and “suppressing” as used herein refer to a course of action (such as administering a pharmaceutical composition) initiated prior to the onset of a clinical manifestation of a disease state or condition so as to reduce the likelihood or severity. Such reduction in likelihood or severity need not be absolute to be useful.

The terms “treatment,” “treat,” and “treating” as used herein refers to a course of action (such as administering a pharmaceutical composition) initiated after the onset of a clinical manifestation of a disease state or condition so as to eliminate or reduce such clinical manifestation of the disease state or condition. Such treating need not be absolute to be useful.

The term “in need of treatment” as used herein refers to a judgment made by a caregiver that a patient requires or will benefit from treatment. This judgment is made based on a variety of factors that are in the realm of a caregiver's expertise, but that include the knowledge that the patient is ill, or will be ill, as the result of a condition that is treatable by a method or device of the present disclosure.

The term “in need of prevention” as used herein refers to a judgment made by a caregiver that a patient requires or will benefit from prevention. This judgment is made based on a variety of factors that are in the realm of a caregiver's expertise, but that include the knowledge that the patient will be ill or may become ill, as the result of a condition that is preventable by a method or device of the disclosure.

The term “individual”, “subject” or “patient” as used herein refers to any animal, including mammals, such as mice, rats, other rodents, rabbits, dogs, cats, swine, cattle, sheep, horses, or primates, and humans. The term may specify male or female or both, or exclude male or female.

The term “therapeutically effective amount” as used herein refers to an amount of a compound, either alone or as a part of a pharmaceutical composition, that is capable of having any detectable, positive effect on any symptom, aspect, or characteristics of a disease state or condition. Such effect need not be absolute to be beneficial.

### NUTRITIONAL COMPOSITIONS

The present disclosure provides a nutritional composition, such as a supplement, for administration to a subject, such as a horse or other animal. The nutritional composition may be useful, for example, in treating and/or preventing airway inflammation diseases and medical conditions in a subject, such as a horse or other animal. In some embodiments, the nutritional composition may be useful in treating and/or preventing Equine Asthma Syndrome (EAS).

The nutritional composition may include one or more polyphenols, such as flavonoids and/or non-flavonoids. In some embodiments, the nutritional composition may include one or more amino acids. In some embodiments, the nutritional composition may include one or more carriers. In some embodiments, the nutritional composition may include one or more additives.

### (1) Polyphenols

In embodiments, the nutritional composition includes one or more polyphenols, such as flavonoids and/or non-flavonoids. The one or more polyphenols may be provided in unencapsulated form or encapsulated form. A polyphenol-based nutritional composition may be useful in treating and/or preventing airway inflammation diseases and medical conditions in animals, such as EAS. Without being bound by a particular theory, the polyphenols may reduce, temper, or mitigate airway inflammation and/or airway inflammatory responses to environmental triggers through antioxidant and anti-inflammatory mechanisms. The anti-inflammatory actions of the polyphenols may be attributed to several mechanisms, including the reduction of inflammatory cytokine production, suppression of oxidative stress, and modulation of the activity of inflammatory transcription factors. As demonstrated herein, in working use cases for horses afflicted by EAS, the polyphenol-based nutritional composition modulates pulmonary inflammation and improves bronchoalveolar neutrophilia and pulmonary pro-inflammatory cytokine production.

### Resveratrol

Resveratrol is an example of a non-flavonoid that may be included in the nutritional composition. Resveratrol is a naturally occurring polyphenol and may have antioxidant and anti-inflammatory properties. Resveratrol may be useful in reducing, tempering, or mitigating airway inflammation and/or airway inflammatory responses to environmental triggers, such as dust, mold, bacterial components, or airborne irritants that may trigger airway inflammation and clinical signs thereof. Accordingly, the nutritional composition may include a therapeutically effective amount of resveratrol for treating and/or preventing airway inflammation and/or airway inflammatory responses to environmental triggers in a patient in need thereof. In some embodiments, resveratrol may be used as a therapeutic agent for treating horses afflicted with EAS. Accordingly, the nutritional composition may include a therapeutically effective amount of resveratrol for treating and/or preventing EAS in a horse in need thereof.

Resveratrol may be provided in unencapsulated form or encapsulated form. Encapsulated resveratrol may be encapsulated in oil, such as grape seed oil, to improve its stability. However, it has been discovered that encapsulated resveratrol may have less bioavailability than unencapsulated resveratrol. Thus, in some embodiments, resveratrol is provided in the nutritional composition in unencapsulated form. The resveratrol may be free or substantially free of a resveratrol carrier (e.g., oil delivery systems).

In some embodiments, resveratrol may be present in the nutritional composition in an amount of at least 0.1 %, by weight of the nutritional composition as a whole (i.e., 0.1% by weight), such as at least 0.2% by weight, at least 0.5% by weight, at least 1% by weight, at least 1.5% by weight, or at least 2% by weight resveratrol. In some embodiments, resveratrol may be present in the nutritional composition in an amount of less than 50% by weight, such as less than 20% by weight, less than 12% by weight, less than 10% by weight, less than 9% by weight, less than 8% by weight, less than 7% by weight, less than 6% by weight, less than 5% by weight, less than 4% by weight, or less than 3% by weight resveratrol. For example, resveratrol may be present in the nutritional composition in an amount between 0.1% by weight to 50% by weight, between 0.1% by weight to 20% by weight, between 0.1% by weight to 15% by weight, between 0.1% by weight to 10% by weight, between 0.1% by weight to 5% by weight, between 0.5% by weight to 20% by weight, between 0.5% by weight to 15% by weight, between 0.5% by weight to 10% by weight, between 0.5% by weight to 5% by weight, between 1% by weight to 20% by weight, between 1% by weight to 10% by weight, between 1% by weight to 5% by weight, between 2% by weight to 20% by weight, between 2% by weight to 10% by weight, between 2% by weight to 5% by weight, between 1% by weight to 4% by weight, between 2% by weight to 4% by weight, between 1% by weight to 2% by weight, between 2% by weight to 3% by weight, or between 3% by weight to 4% by weight resveratrol, or any subrange thereof including any combination of the upper and lower ranges set forth above. In embodiments, resveratrol may be present in the nutritional composition in an amount included in any range set forth above. For example, resveratrol may be present in the nutritional composition in an amount of about 0.5% by weight, about 0.75% by weight, about 1% by weight, about 1.25% by weight, about 1.5% by weight, about 1.75% by weight, about 2% by weight, about 2.25% by weight, about 2.5% by weight, about 2.75% by weight, about 3% by weight, about 3.25% by weight, about 3.5% by weight, about 3.75% by weight, about 4% by weight, about 4.25% by weight, about 4.5% by weight, or about 4.75% by weight resveratrol.

### Citrus Bioflavonoids

Citrus bioflavonoids are an example of a flavonoid that may be included in the nutritional composition. Citrus bioflavonoids are naturally occurring polyphenols that may be derived from citrus fruits or plants, such as oranges, mandarins, grapefruit, lemons, limes, etc. Citrus bioflavonoids may have antioxidant and anti-inflammatory properties that may be useful in reducing, tempering, or mitigating airway inflammation and/or airway inflammatory responses to environmental triggers. Accordingly, the nutritional composition may include a therapeutically effective amount of citrus bioflavonoid for treating and/or preventing airway inflammation and/or airway inflammatory responses to environmental triggers in a patient in need thereof. In some embodiments, citrus bioflavonoid may be used as a therapeutic agent for treating horses afflicted with EAS. Accordingly, the nutritional composition may include a therapeutically effective amount of citrus bioflavonoid for treating and/or preventing EAS in a horse in need thereof.

In some embodiments, citrus bioflavonoid, meaning one or more citrus bioflavonoids, may be present in the nutritional composition in an amount of at least 0.1% by weight of the nutritional composition as a whole (i.e., 0.1% by weight), such as at least 1% by weight, at least 5% by weight, at least 10% by weight, at least 12% by weight, or at least 15% by weight citrus bioflavonoid. In some embodiments, citrus bioflavonoid may be present in the nutritional composition in an amount of less than 50% by weight, such as less than 45% by weight, less than 40% by weight, less than 35% by weight, less than 30% by weight, less than 25% by weight, or less than 20% by weight citrus bioflavonoid. For example, citrus bioflavonoid may be present in the nutritional composition in an amount between 0.1% by weight to 50% by weight, between 5% by weight to 50% by weight, between 5% by weight to 40% by weight, between 5% by weight to 30% by weight, between 5% by weight to 25% by weight, between 5% by weight to 20% by weight, between 5% by weight to 15% by weight, between 10% by weight to 50% by weight, between 10% by weight to 40% by weight, between 10% by weight to 30% by weight, between 10% by weight to 25% by weight, between 10% by weight to 20% by weight, between 15% by weight to 50% by weight, between 15% by weight to 40% by weight, between 15% by weight to 30% by weight, between 15% by weight to 25% by weight, between 15% by weight to 20% by weight, between 20% by weight to 40% by weight, or between 20% by weight to 30% by weight citrus bioflavonoid, or any subrange thereof including any combination of the upper and lower ranges set forth above. In embodiments, citrus bioflavonoid may be present in the nutritional composition in an amount included in any range set forth above. For example, citrus bioflavonoid may be present in the nutritional composition in an amount of about 1% by weight, about 5% by weight, about 7.5% by weight, about 9% by weight, about 10% by weight, about 12.5% by weight, about 15% by weight, about 17.5% by weight, about 18% by weight, about 20% by weight, about 22.5% by weight, about 25% by weight, about 27.5% by weight, or about 30% by weight citrus bioflavonoid.

### Hesperidin

Hesperidin is another example of a flavonoid that may be included in the nutritional composition. Hesperidin is a naturally occurring polyphenol that may be derived from citrus fruits or plants, such as oranges, mandarins, grapefruit, lemons, limes, etc. Hesperidin may have antioxidant and anti-inflammatory properties that may be useful in reducing, tempering, or mitigating airway inflammation and/or airway inflammatory responses to environmental triggers. Accordingly, the nutritional composition may include a therapeutically effective amount of hesperidin for treating and/or preventing airway inflammation and/or airway inflammatory responses to environmental triggers in a patient in need thereof. In some embodiments, hesperidin may be used as a therapeutic agent for treating horses afflicted with EAS. Accordingly, the nutritional composition may include a therapeutically effective amount of hesperidin for treating and/or preventing EAS in a horse in need thereof.

In some embodiments, hesperidin may be present in the nutritional composition in an amount of at least 0.1 %, by weight of the nutritional composition as a whole (i.e., 0.1% by weight), such as at least 0.5% by weight, at least 1% by weight, at least 2% by weight, at least 3% by weight, or at least 4% by weight hesperidin. In some embodiments, hesperidin may be present in the nutritional composition in an amount of less than 50% by weight, such as less than 20% by weight, less than 15% by weight, less than 12% by weight, less than 10% by weight, less than 7% by weight, or less than 5% by weight hesperidin. For example, hesperidin may be present in the nutritional composition in an amount between 0.1% by weight to 50% by weight, between 1% by weight to 20% by weight, between 1% by weight to 15% by weight, between 1% by weight to 10% by weight, between 1% by weight to 7% by weight, between 1% by weight to 5% by weight, between 1% by weight to 4% by weight, between 2% by weight to 20% by weight, between 2% by weight to 15% by weight, between 2% by weight to 10% by weight, between 2% by weight to 7% by weight, between 2% by weight to 5% by weight, between 3% by weight to 20% by weight, between 3% by weight to 15% by weight, between 3% by weight to 10% by weight, between 3% by weight to 7% by weight, between 3% by weight to 5% by weight, between 4% by weight to 20% by weight, between 4% by weight to 15% by weight, between 4% by weight to 10% by weight, between 4% by weight to 7% by weight, or between 4% by weight to 5% by weight hesperidin, or any subrange thereof including any combination of the upper and lower ranges set forth above. In embodiments, hesperidin may be present in the nutritional composition in an amount included in any range set forth above. For example, hesperidin may be present in the nutritional composition in an amount of about 0.5% by weight, about 1% by weight, about 1.5% by weight, about 2% by weight, about 2.5% by weight, about 3% by weight, about 3.5% by weight, about 4% by weight, about 4.5% by weight, about 5% by weight, about 5.5% by weight, or about 6% by weight hesperidin.

### Quercetin

Quercetin is another example of a flavonoid that may be included in the nutritional composition. Quercetin is a naturally occurring polyphenol that may be derived from various fruits, vegetables, leaves, seed, and grains, for example, capers, red onions, and kale. Quercetin may have antioxidant and anti-inflammatory properties that may be useful in reducing, tempering, or mitigating airway inflammation and/or airway inflammatory responses to environmental triggers. Accordingly, the nutritional composition may include a therapeutically effective amount of quercetin for treating and/or preventing airway inflammation and/or airway inflammatory responses to environmental triggers in a patient in need thereof. In some embodiments, quercetin may be used as a therapeutic agent for treating horses afflicted with EAS. Accordingly, the nutritional composition may include a therapeutically effective amount of quercetin for treating and/or preventing EAS in a horse in need thereof.

In some embodiments, quercetin may be present in the nutritional composition in an amount of at least 0.1 % by weight of the nutritional composition as a whole (i.e., 0.1% by weight), such as at least 0.25% by weight, at least 0.5% by weight, at least 0.75% by weight, at least 1% by weight, at least 1.25% by weight, or at least 1.5% by weight quercetin. In some embodiments, quercetin may be present in the nutritional composition in an amount of less than 50% by weight, such as less than 10% by weight, less than 7% by weight, less than 5% by weight, less than 4% by weight, less than 3% by weight, or less than 2% by weight quercetin. For example, quercetin may be present in the nutritional composition in an amount between 0.1% by weight to 50% by weight, between 0.1% by weight to 10% by weight, between 0.1% by weight to 5% by weight, between 0.1% by weight to 4% by weight, between 0.1% by weight to 3% by weight, between 0.1% by weight to 2% by weight, between 0.5% by weight to 10% by weight, between 0.5% by weight to 5% by weight, between 0.5% by weight to 4% by weight, between 0.5% by weight to 3% by weight, between 0.5% by weight to 2% by weight, between 0.5% by weight to 1.5% by weight, between 1% by weight to 10% by weight, between 1% by weight to 5% by weight, between 1% by weight to 4% by weight, between 1% by weight to 3% by weight, between 1% by weight to 10% by weight, between 1% by weight to 5% by weight, between 1% by weight to 4% by weight, between 1% by weight to 3% by weight, between 1% by weight to 2% by weight, between 1.5% by weight to 10% by weight, between 1.5% by weight to 5% by weight, between 1.5% by weight to 4% by weight, between 1.5% by weight to 3% by weight, between 1.5% by weight to 2% by weight, or between 2% by weight to 3% by weight quercetin, or any subrange thereof including any combination of the upper and lower ranges set forth above. In embodiments, quercetin may be present in the nutritional composition in an amount included in any range set forth above. For example, quercetin may be present in the nutritional composition in an amount of about 0.1% by weight, about 0.5% by weight, about 0.75% by weight., about 1% by weight, about 1.25% by weight, about 1.5% by weight, about 1.75% by weight, about 2% by weight, about 2.25% by weight, about 2.5% by weight, about 2.75% by weight, or about 3% by weight quercetin.

### (2) Amino Acids

The nutritional composition may include one or more amino acids, such as branched chain amino acids. In some embodiments, the amino acid, meaning one or more amino acids, included in the nutritional composition may be an essential amino acid in the diet of the patient, such as a horse or other animal. The nutritional composition may include essential amino acid that the patient cannot naturally synthesize and must ingest from a supplemental source.

### Leucine

Leucine is an example of an amino acid that may be included in the nutritional composition. Leucine is a branched chain amino acid and is an essential amino acid in the diet of animals, as animals cannot synthesize it and must ingest it from other sources. Leucine may be provided in the nutritional composition in a free form—that is, not provided in proteins or as part of a polypeptide chain. Some embodiments of the nutritional composition include leucine provided as part of proteins and/or polypeptide chains.

In some embodiments, leucine may be present in the nutritional composition in an amount of at least 0.1% by weight of the nutritional composition as a whole (i.e., 0.1% by weight), such as at least 0.5% by weight, at least 1% by weight, at least 2% by weight, at least 3% by weight, at least 4% by weight, or at least 5% by weight leucine. In some embodiments, leucine may be present in the nutritional composition in an amount of less than 50% by weight, such as less than 20% by weight, less than 15% by weight, less than 12% by weight, less than 10% by weight, less than 7% by weight, or less than 6% by weight leucine. For example, leucine may be present in the nutritional composition in an amount between 0.1% by weight to 50% by weight, between 1% by weight to 20% by weight, between 1% by weight to 15% by weight, between 1% by weight to 10% by weight, between 1% by weight to 7% by weight, between 1% by weight to 6% by weight, between 1% by weight to 5% by weight, between 1% by weight to 4% by weight, between 1% by weight to 3% by weight, between 2% by weight to 20% by weight, between 2% by weight to 15% by weight, between 2% by weight to 10% by weight, between 2% by weight to 7% by weight, between 2% by weight to 6% by weight, between 3% by weight to 20% by weight, between 3% by weight to 15% by weight, between 3% by weight to 10% by weight, between 3% by weight to 7% by weight, between 3% by weight to 6% by weight, between 4% by weight to 20% by weight, between 4% by weight to 15% by weight, between 4% by weight to 10% by weight, between 4% by weight to 7% by weight, between 4% by weight to 6% by weight, between 5% by weight to 6% by weight, or between 6% by weight to 7% by weight leucine, or any subrange thereof including any combination of the upper and lower ranges set forth above. In embodiments, leucine may be present in the nutritional composition in an amount included in any range set forth above. For example, leucine may be present in the nutritional composition in an amount of about 0.5% by weight, about 1% by weight, about 1.5% by weight, about 2% by weight, about 2.5% by weight, about 2.75% by weight, about 3% by weight, about 3.5% by weight, about 4% by weight, about 4.5% by weight, about 5% by weight, about 5.5% by weight, about 6% by weight, about 6.5% by weight, or about 7% by weight leucine.

### Lysine

Lysine is another example of an amino acid that may be included in the nutritional composition. Lysine is a branched chain amino acid and is an essential amino acid in the diet of animals, as animals cannot synthesize it and must ingest it from other sources. Lysine may be provided in the nutritional composition in a free form—that is, not provided in proteins or as part of a polypeptide chain. Some embodiments of the nutritional composition include lysine provided as part of proteins and/or polypeptide chains.

In some embodiments, lysine may be present in the nutritional composition in an amount of at least 0.1% by weight of the nutritional composition as a whole (i.e., 0.1% by weight), such as at least 0.5% by weight, at least 1% by weight, at least 2% by weight, at least 3% by weight, at least 4% by weight, or at least 5% by weight lysine. In some embodiments, lysine may be present in the nutritional composition in an amount of less than 50% by weight, such as less than 20% by weight, less than 15% by weight, less than 12% by weight, less than 10% by weight, less than 7% by weight, or less than 6% by weight lysine. For example, lysine may be present in the nutritional composition in an amount between 0.1% by weight to 50% by weight, between 1% by weight to 20% by weight, between 1% by weight to 15% by weight, between 1% by weight to 10% by weight, between 1% by weight to 7% by weight, between 1% by weight to 6% by weight, between 2% by weight to 20% by weight, between 2% by weight to 15% by weight, between 2% by weight to 10% by weight, between 2% by weight to 7% by weight, between 2% by weight to 6% by weight, between 2% by weight to 5% by weight, between 2% by weight to 4% by weight, between 3% by weight to 20% by weight, between 3% by weight to 15% by weight, between 3% by weight to 10% by weight, between 3% by weight to 7% by weight, between 3% by weight to 6% by weight, between 4% by weight to 20% by weight, between 4% by weight to 15% by weight, between 4% by weight to 10% by weight, between 4% by weight to 7% by weight, between 4% by weight to 6% by weight, between 5% by weight to 6% by weight, or between 6% by weight to 7% by weight lysine, or any subrange thereof including any combination of the upper and lower ranges set forth above. In embodiments, lysine may be present in the nutritional composition in an amount included in any range set forth above. For example, lysine may be present in the nutritional composition in an amount of about 0.5% by weight, about 1% by weight, about 1.5% by weight, about 2% by weight, about 2.5% by weight, about 3% by weight, about 3.5% by weight, about 4% by weight, about 4.5% by weight, about 5% by weight, about 5.5% by weight, about 6% by weight, about 6.5% by weight, or about 7% by weight lysine.

### Additional Amino Acids

Embodiments of the nutritional composition may include one or more additional amino acids, including one or more of alanine, arginine, asparagine, aspartic acid, cysteine, glutamine, glutamic acid, glycine, histidine, isoleucine, methionine, phenylalanine, proline, serine, threonine, tryptophan, tyrosine, or valine. In some embodiments, one or more of alanine, glutamic acid, glycine, or proline are included in the nutritional composition. The additional amino acid, meaning one or more additional amino acids, may be provided in free form or as part of proteins and/or polypeptide chains.

In some embodiments, each additional amino acid, meaning each of the one or more amino acids that is present in the nutritional composition, may be present in the nutritional composition in an amount of at least 0.1% by weight of the composition as a whole (i.e., 0.1% by weight), such as at least 0.25% by weight, at least 0.5% by weight, at least 0.75% by weight, at least 1% by weight, at least 1.25% by weight, or at least 1.5% by weight of the additional amino acid. In some embodiments, each additional amino acid may be present in the nutritional composition in an amount of less than 50% by weight, such as less than 10% by weight, less than 7% by weight, less than 5% by weight, less than 4% by weight, less than 3% by weight, or less than 2% by weight of the additional amino acid. For example, each additional amino acid may be present in the nutritional composition in an amount between 0.1% by weight to 50% by weight, between 0.1% by weight to 10% by weight, between 0.1% by weight to 5% by weight, between 0.1% by weight to 4% by weight, between 0.1% by weight to 3% by weight, between 0.1% by weight to 2% by weight, between 0.5% by weight to 10% by weight, between 0.5% by weight to 5% by weight, between 0.5% by weight to 4% by weight, between 0.5% by weight to 3% by weight, between 0.5% by weight to 2% by weight, between 0.5% by weight to 1% by weight, between 1% by weight to 10% by weight, between 1% by weight to 5% by weight, between 1% by weight to 4% by weight, between 1% by weight to 3% by weight, between 1% by weight to 10% by weight, between 1% by weight to 5% by weight, between 1% by weight to 4% by weight, between 1% by weight to 3% by weight, between 1% by weight to 2% by weight, between 1.5% by weight to 10% by weight, between 1.5% by weight to 5% by weight, between 1.5% by weight to 4% by weight, between 1.5% by weight to 3% by weight, between 1.5% by weight to 2% by weight, between 1% by weight to 1.5% by weight, or between 2% by weight to 3% by weight of the additional amino acid, or any subrange thereof including any combination of the upper and lower ranges set forth above. In embodiments, each additional amino acid may be present in the nutritional composition in an amount included in any range set forth above. For example, each additional amino acid may be present in the nutritional composition in an amount of about 0.1% by weight, 0.5% by weight, about 0.75% by weight., about 1% by weight, about 1.25% by weight, about 1.5% by weight, about 1.75% by weight, about 2% by weight, about 2.25% by weight, about 2.5% by weight, about 2.75% by weight, or about 3% by weight of the additional amino acid.

### (3) Carriers

The nutritional composition may include a carrier. In some embodiments, the carrier may be suitable for granular or powder-type formulations of the nutritional composition. For example, the carrier may include yeast and/or oil. In some embodiments, the carrier may be suitable for pellet-type formulations of the nutritional composition. For example, the carrier may include yeast, plant meal such as alfalfa meal, grain, grain byproducts, rice bran, and the like. In some embodiments, the carrier includes active dry yeast, such as dried yeast granules or dried yeast fermentation solubles. In various embodiments, the carrier may include yeast, oil, plant meal such as alfalfa meal, grain, grain byproducts, rice bran, or any other suitable carrier for use in oral ingestion type formulations. The carrier, meaning one or more carriers, may facilitate delivery of the polyphenols, amino acids, and/or additives of the nutritional composition for ingestion by the animal.

In some embodiments, the carrier may be present in the nutritional composition in an amount of at least 10% by weight of the composition as a whole (i.e., 10% by weight), such as at least 20% by weight, at least 30% by weight, at least 40% by weight, at least 50% by weight, or at least 60% by weight carrier. In some embodiments, the carrier may be present in the nutritional composition in an amount of less than 99% by weight, such as less than 95% by weight, less than 90% by weight, less than 85% by weight, less than 80% by weight, less than 75% by weight, or less than 70% by weight carrier. For example, the carrier may be present in the nutritional composition in an amount between 10% by weight to 99% by weight, between 20% by weight to 99% by weight, between 30% by weight to 99% by weight, between 40% by weight to 99% by weight, between 50% by weight to 99% by weight, between 60% by weight to 99% by weight, between 50% by weight to 95% by weight, between 50% by weight to 90% by weight, between 50% by weight to 85% by weight, between 50% by weight to 80% by weight, between 50% by weight to 75% by weight, between 50% by weight to 70% by weight, between 60% by weight to 95% by weight, between 60% by weight to 90% by weight, between 60% by weight to 85% by weight, between 60% by weight to 80% by weight, between 60% by weight to 75% by weight, between 60% by weight to 70% by weight carrier, between 70% by weight to 80% by weight, or any subrange thereof including any combination of the upper and lower ranges set forth above. In embodiments, the carrier may be present in the nutritional composition in an amount included in any range set forth above. For example, the carrier may be present in the nutritional composition in an amount of about 50% by weight, about 55% by weight, about 60% by weight, about 65% by weight, or about 70% by weight carrier.

### (4) Additives

The nutritional composition may include one or more additives that may impart desired properties on the nutritional composition. Examples of additives include vehicles, adjuvants, surfactants, suspending agents, emulsifying agents, inert fillers, diluents, excipients, wetting agents, binders, lubricants, buffering agents, disintegrating agents and carriers, accessory agents, such as coloring agents, flavoring agents, and/or sweetening agents, water, pH buffers, humectants, thickeners, gums, antibacterial agents, grain, molasses, salt, among others. The additives included in the nutritional composition may vary depending on the intended method of administering the nutritional composition, the patient, etc.

### Accessory Agents

In some embodiments, the nutritional composition includes one or more accessory agents, such as coloring agents, flavoring agents, and/or sweetening agents. The accessory agent may impart desired properties that make the ingestion of the nutritional composition more pleasant for the patient. For example, the accessory agents may impart a taste to the nutritional composition that makes the ingestion of the nutritional composition more pleasant for an animal such as a horse. Examples of coloring agents include food dyes. Examples of flavoring agents may include one or more of water-soluble essential oils, apple, banana, cherry, carrot, cumin, fenugreek, fruit punch, rosemary, peppermint, oregano, and the like. Examples of sweetening agents include sweet doux, saccharin, dextrose, levulose, cyclamate, aspartate, and the like.

In some embodiments, each accessory agent, meaning each of the one or more accessory agents that is present in the nutritional composition, may be present in the nutritional composition in an amount of at least 0.01% by weight of the composition as a whole (i.e., 0.01% by weight), such as at least 0.025% by weight, at least 0.05% by weight, at least 0.075% by weight, at least 0.1% by weight, at least 0.125% by weight, or at least 0.15% by weight of the accessory agent. In some embodiments, each accessory agent may be present in the nutritional composition in an amount of less than 5% by weight, such as less than 1% by weight, less than 0.75% by weight, less than 0.5% by weight, less than 0.4% by weight, less than 0.3% by weight, or less than 0.2% by weight of the accessory agent. For example, each accessory agent may be present in the nutritional composition in an amount between 0.01% by weight to 5% by weight, between 0.01% by weight to 1% by weight, between 0.01% by weight to 0.5% by weight, between 0.01% by weight to 0.4% by weight, between 0.01% by weight to 0.3% by weight, between 0.01% by weight to 0.2% by weight, between 0.05% by weight to 1% by weight, between 0.05% by weight to 0.5% by weight, between 0.05% by weight to 0.4% by weight, between 0.05% by weight to 0.3% by weight, between 0.05% by weight to 0.2% by weight, between 0.1% by weight to 1% by weight, between 0.1% by weight to 0.5% by weight, between 0.1% by weight to 0.4% by weight, between 0.1% by weight to 0.3% by weight, or between 0.1% by weight to 0.2% by weight of the accessory agent, or any subrange thereof including any combination of the upper and lower ranges set forth above. In embodiments, each accessory agent may be present in the nutritional composition in an amount included in any range set forth above. For example, each accessory agent may be present in the nutritional composition in an amount of about 0.01% by weight, 0.05% by weight, about 0.075% by weight., about 0.1% by weight, about 0.125% by weight, about 0.15% by weight, about 0.175% by weight, or about 0.2% by weight of the accessory agent.

### Humectants

In some embodiments, the nutritional composition may include one or more humectants. The humectant, meaning one or more humectants, may prevent dry-out and impart pleasant mouth feel to the nutritional composition when ingested. Examples of humectants include glycerin, sorbitol, polypropylene glycol, xylitol, and polyethylene glycol.

### Thickeners

In some embodiments, the nutritional composition may include one or more thickeners. Examples of thickeners include silica thickeners, sodium aluminum silicates, and clays.

### Gums

In some embodiments, the nutritional composition may include one or more gums. Examples of gums include sodium carboxymethyl cellulose, cellulose ethers, xantham gum, carrageenans, sodium alginate, and carbopols.

### Example Nutritional Compositions

The nutritional compositions described herein may be useful for treating and/or preventing airway inflammation diseases and medical conditions in a patient in need thereof. In some embodiments, the nutritional composition may be useful for treating and/or preventing EAS in a horse in need thereof, such as a horse afflicted by EAS. In some embodiments, the nutritional composition may be used in granular or powder-type formulations. In some embodiments, the nutritional composition may be used in pellet-type formulations. The nutritional composition may be in any suitable formulation. In some embodiments, the nutritional composition may be in a formulation suitable for oral ingestions.

An example of a nutritional composition for treating and/or preventing airway inflammation diseases and medical conditions in a patient in need thereof includes one or more polyphenols. The nutritional composition may be useful for treating and/or preventing EAS in a horse in need thereof, such as a horse afflicted by EAS. The nutritional composition may include any combination of the polyphenols described above. The polyphenols may be present in the nutritional composition in any of the amounts described above in any combination. In some embodiments, the nutritional composition may include one of the polyphenols described above, in any of the amounts described above for the polyphenol. In some embodiments, the nutritional composition may include each of the polyphenols described above, and each polyphenol may be present in any of the amounts described above for the polyphenol. In some embodiments, the nutritional composition may include a sub-combination of the polyphenols described above, such as more than one and fewer than all the polyphenols, e.g., two of the polyphenols or three of the polyphenols, and each polyphenol of the sub-combination may be present in any of the amounts described above for the polyphenol. The nutritional composition may include a therapeutically effective amount of one or more phenols.

In some embodiments, the one or more polyphenols in the nutritional composition includes resveratrol in an amount between 0.5% by weight of the nutritional composition as a whole (i.e., 0.5% by weight) to 10% by weight, such as between 1% by weight to 5% by weight, between 1% by weight to 4% by weight, or between 1% by weight to 3% by weight; citrus bioflavonoid in an amount between 5% by weight to 30% by weight, such as between 5% by weight to 25% by weight, or between 5% by weight to 20% by weight; hesperidin in an amount between 1% by weight to 10% by weight, such as between 1% by weight to 5% by weight, or between 2% by weight to 5% by weight; and/or quercetin in an amount between 0.1% by weight to 5% by weight, such as between 0.5% by weight to 5% by weight, between 0.5% by weight to 3% by weight, or between 0.5% by weight to 2% by weight; and any combination thereof.

In some embodiments, the one or more polyphenols in the nutritional composition includes resveratrol in an amount of about 0.5% by weight of the nutritional composition as a whole (i.e., 0.5% by weight), about 0.75% by weight, about 1% by weight, about 1.25% by weight, about 1.5% by weight, about 1.75% by weight, about 2% by weight, about 2.25% by weight, about 2.5% by weight, about 2.75% by weight, or about 3% by weight; citrus bioflavonoid in an amount of about 7.5% by weight, about 9% by weight, about 10% by weight, about 12.5% by weight, about 15% by weight, about 17% by weight, about 17.5% by weight, about 18% by weight, about 18.5% by weight, or about 19% by weight; hesperidin in an amount of about 2% by weight, about 2.25% by weight, about 2.5% by weight, about 2.75% by weight, about 3% by weight, about 3.25% by weight, about 3.5% by weight, about 3.75% by weight, about 4% by weight, about 4.25% by weight, about 4.5% by weight, about 4.75% by weight, or about 5% by weight; and/or quercetin in an amount of about 0.5% by weight, about 0.75% by weight, about 1% by weight, about 1.25% by weight, about 1.5% by weight, about 1.75% by weight, or about 2% by weight; and any combination thereof.

In some embodiments, the nutritional composition includes the one or more polyphenols and one or more amino acids. The nutritional composition may include any combination of the amino acids described above. The amino acids may be present in the nutritional composition in any of the amounts described above in any combination. In some embodiments, the nutritional composition may include one of the amino acids described above, in any of the amounts described above for the amino acid.

In some embodiments, the one or more amino acids included in the nutritional composition may include lysine and/or leucine. Lysine and/or leucine may be present in any of the respective amounts described above. In some embodiments, the nutritional composition may include lysine and/or leucine and one or more of the additional amino acids described above, and each amino acid may be present in any of the amounts described above for the amino acid. In some embodiments, the nutritional composition may include a sub-combination of the amino acids described above, such as more than one and fewer than all the amino acids, and each amino acid of the sub-combination may be present in any of the amounts respectively described above for the amino acid.

In some embodiments, the one or more amino acids in the nutritional composition includes leucine in an amount between 0.1% by weight of the nutritional composition as a whole (i.e., 0.1% by weight) to 20% by weight, such as between 1% by weight to 10% by weight, between 2% by weight to 7% by weight, between 2% by weight to 6% by weight, between 2% by weight to 5% by weight, between 5% by weight to 7% by weight, between 5% by weight to 6% by weight, or between 6% by weight to 7% by weight; and/or lysine in an amount between 0.1% by weight to 20% by weight, such as between 1% by weight to 10% by weight, between 2% by weight to 7% by weight, between 2% by weight to 6% by weight, between 2% by weight to 5% by weight, between 5% by weight to 7% by weight, between 5% by weight to 6% by weight, or between 6% by weight to 7% by weight; optionally, one or more additional amino acids each in an amount between 0.1% by weight to 5% by weight, between such as between 0.1% by weight to 3% by weight, between 0.1% by weight to 2% by weight, between 0.1% by weight to 1% by weight, between 1% by weight to 2% by weight, or between 2% by weight to 3% by weight; and any combination thereof.

In some embodiments, the one or more amino acids in the nutritional composition includes leucine in an amount of about 1% by weight of the nutritional composition as a whole (i.e., 1% by weight), about 1.5% by weight, about 2% by weight, about 2.5% by weight, about 3% by weight, about 3.5% by weight, about 4% by weight, about 4.5% by weight, about 5% by weight, about 5.5% by weight, about 6% by weight, about 6.5% by weight, or about 7% by weight; and/or lysine in an amount of about 1% by weight, about 1.5% by weight, about 2% by weight, about 2.5% by weight, about 3% by weight, about 3.5% by weight, about 4% by weight, about 4.5% by weight, about 5% by weight, about 5.5% by weight, about 6% by weight, about 6.5% by weight, or about 7% by weight; and any combination thereof.

In some embodiments, the nutritional composition may include a carrier, such as one or more carriers. The carrier may include active dry yeast, such as dried yeast granules or dried yeast fermentation solubles. In some embodiments, the nutritional composition includes the one or more polyphenols and the carrier. In some embodiments, the nutritional composition includes the one or more polyphenols, the one or more amino acids, and the carrier.

The carrier may be present in the nutritional composition in any of the amounts described above. In some embodiments, the carrier may be present in the nutritional composition in an amount between 10% by weight of the nutritional composition as a whole (i.e., 10% by weight) to 99% by weight, such as between 50% by weight to 99% by weight, between 60% by weight to 99% by weight, between 60% by weight to 90% by weight, between 60% by weight to 75% by weight, between 60% by weight to 70%, or between 70% to 80% by weight carrier.

In some embodiments, the nutritional composition may include an additive, such as one or more additives. The additive may include one or more accessory agents, such as coloring agents, flavoring agents, and/or sweetening agents. In some embodiments, the nutritional composition includes the one or more polyphenols and the additive. In some embodiments, the nutritional composition includes the one or more polyphenols, the one or more amino acids, and the additive. In some embodiments, the nutritional composition includes the one or more polyphenols, the carrier, and the additive. In some embodiments, the nutritional composition includes the one or more polyphenols, the one or more amino acids, the carrier, and the additive.

The additive may be present in the nutritional composition in any of the amounts described above. In some embodiments, the additive may be present in the nutritional composition in an amount between 0.01% by weight of the nutritional composition as a whole (i.e., 0.01% by weight) to 5% by weight, between 0.05% by weight to 1% by weight, between 0.05% by weight to 0.5% by weight, or between 0.05% by weight to 0.2% by weight.

### FORMULATIONS AND METHODS OF ADMINISTERING

The nutritional composition may be formulated as a liquid, a gel, a pill form, a pellet, or in a dry granular or powder form. Embodiments provided in a pellet form or a dry granular or powder form may be particularly advantageous, as these embodiments may allow for the nutritional composition to be mixed with food from an animal's normal diet, such as commercially available horse feed, to enable convenient administration to the animal. Beneficially, mixing the nutritional composition with food from an animal's diet may improve digestion and bioabsorption of the nutritional composition in the animal. The nutritional composition may be provided as a concentrate. The nutritional composition may be provided in scoopable form (e.g., granular) for periodic or single dose administration to the animal.

In some embodiments, the nutritional composition may be provided in a form for daily administration. For example, the nutritional composition may be provided in an amount of at least 2 grams (g), such as at least 5 g, at least 10 g, at least 20 g, or at least 25 g. In some embodiments, the nutritional composition for daily administration is provided in an amount of less than 1000 g, such as less than 500 g, less than 250 g, less than 200 g, less than 150 g, less than 125 g, less than 100 g, less than 75 g, less than 50 g, less than 40 g, or less than 30 g. In some embodiments, the nutritional composition for daily administration is provided in an amount of between 2 g to 1000 g, between 2 g to 500 g, between 2 g to 250 g, between 2 g to 100 g, between 2 g to 50 g, between 2 g to 30 g, between 5 g to 250 g, between 5 g to 100 g, between 5 g to 50 g, between 5 g to 30 g, between 10 g to 250 g, between 10 g to 100 g, between 10 g to 50 g, between 50g to 100g, between 50g to 75g, between 10 g to 30 g, between 20 g to 50 g, or between 20 g to 30 g, or any subrange thereof including any combination of the upper and lower ranges set forth above. In embodiments, the nutritional composition for daily administration is provided in an amount included in any range set forth above. For example, the nutritional composition for daily administration is provided in an amount of about 2 g, about 5 g, about 10 g, about 15 g, about 20 g, about 25 g, about 30 g, about 35 g, about 40 g, about 45 g, about 50 g, about 55g, about 60g, or about 65g.

In some embodiments, the nutritional composition may be provided in a form for twice-daily administration. For example, the nutritional composition may be provided in an amount of at least 1 g, such as at least 2 g, at least 5 g, or at least 10 g. In some embodiments, the nutritional composition for twice-daily administration is provided in an amount of less than 1000 g, such as less than 500 g, less than 250 g, less than 200 g, less than 150 g, less than 125 g, less than 100 g, less than 75 g, less than 50 g, less than 40 g, less than 30 g, or less than 15 g. In some embodiments, the nutritional composition for twice-daily administration is provided in an amount of between 1 g to 1000 g, between 2 g to 500 g, between 2 g to 250 g, between 2 g to 100 g, between 2 g to 50 g, between 2 g to 30 g, between 2 g to 15 g, between 5 g to 100 g, between 5 g to 50 g, between 5 g to 30 g, between 5 g to 15 g, between 10 g to 100 g, between 10 g to 50 g, between 10 g to 30 g, between 20g to 30g, between 10 g to 25 g, or between 10 g to 15 g, or any subrange thereof including any combination of the upper and lower ranges set forth above. In embodiments, the nutritional composition for twice-daily administration is provided in an amount included in any range set forth above. For example, the nutritional composition for twice-daily administration is provided in an amount of about 1 g, about 2 g, about 5 g, about 7 g, about 10 g, about 12 g, about 14 g, about 15 g, about 20 g, about 25 g, about 30 g, or about 35 g.

In embodiments, a method for treating or preventing an airway inflammation disease or medical condition in an animal, such as an equine, in need thereof is provided. The method may include administering to the equine the nutritional composition described herein. The nutritional composition may be administered daily or twice-daily, in the amounts described above. In some embodiments, the nutritional composition may include a therapeutically effective amount of one or more phenols. The one or more phenols may include flavonoids and/or non-flavonoids. In some embodiments, the one or more phenols may include resveratrol, citrus bioflavonoid, hesperidin, and/or quercetin. In some embodiments, the nutritional composition may include one or more amino acids, such as lysine, leucine, and/or one or more additional amino acids. In some embodiments, the nutritional composition may include a carrier and/or an additive. The aforementioned ingredients may be provided in their respective amounts described herein.

In embodiments, a method for treating or preventing airway inflammatory responses to environmental triggers, such as dust, mold, bacterial components, or airborne irritants, in an animal, such as an equine, in need thereof is provided. The method may include administering to the equine the nutritional composition described herein. The nutritional composition may be administered daily or twice-daily, in the amounts described above. In some embodiments, the nutritional composition may include a therapeutically effective amount of one or more phenols. The one or more phenols may include flavonoids and/or non-flavonoids. In some embodiments, the one or more phenols may include resveratrol, citrus bioflavonoid, hesperidin, and/or quercetin. In some embodiments, the nutritional composition may include one or more amino acids, such as lysine, leucine, and/or one or more additional amino acids. In some embodiments, the nutritional composition may include a carrier and/or an additive. The aforementioned ingredients may be provided in their respective amounts described herein.

In embodiments, a method for treating or preventing equine asthma syndrome (EAS) in an equine in need thereof is provided. The method may include administering to the equine the nutritional composition described herein. The nutritional composition may be administered daily or twice-daily, in the amounts described above. In some embodiments, the nutritional composition may include a therapeutically effective amount of one or more phenols. The one or more phenols may include flavonoids and/or non-flavonoids. In some embodiments, the one or more phenols may include resveratrol, citrus bioflavonoid, hesperidin, and/or quercetin. In some embodiments, the nutritional composition may include one or more amino acids, such as lysine, leucine, and/or one or more additional amino acids. In some embodiments, the nutritional composition may include a carrier and/or an additive. The aforementioned ingredients may be provided in their respective amounts described herein.

In the methods described herein, the therapeutically effective amount of the one or more phenols in the administered nutritional composition may vary according to a variety of factors such as the subject's condition, weight, sex, and age. For example, some embodiments of the composition comprise up to the median lethal dose (LD50) of the active compound. The LD50 can be ascertained using standard toxicological methods, or by reference to past studies.

The methods described herein may include enteral (e.g., oral) administration of the nutritional composition to the animal. The nutritional composition may be orally administered to the animal in daily dose form or twice-daily dose form, in the amounts described herein. In some embodiments, the method may include contacting, or mixing, the nutritional composition with animal feed, such as equine feed, prior to administration. As used herein, the term “feed” refers to a composition for generally meeting the basic nutritional needs of an animal.

The methods described herein may include administering the nutritional composition to an equine. In some embodiments, the methods described herein may include administering the nutritional composition to an animal other than an equine, such as, for example, a mammal, an ungulate, a pig, a camel, a deer, a mule, or a donkey.

### EXAMPLES

Aspects of the present disclosure will now be described with reference to non-limiting examples.

Working Example 1—Study on the effects of polyphenol supplementation on lower airway inflammation in horses with Equine Asthma.

### Introduction

Equine asthma is a syndrome of non-infectious lower airway inflammation that commonly affects horses of all ages around the world. See Couëtil, L.L., et al. “Inflammatory Airway Disease of Horses—Revised Consensus Statement.” Journal of Veterinary Internal Medicine, vol. 30, no. 2, 2016, pp. 503-515., https: //doi. org/10.1111/jvim.13824 (“Couetil et al.”); Davis KU and Sheats MK (2019) Bronchoalveolar Lavage Cytology Characteristics and Seasonal Changes in a Herd of Pastured Teaching Horses. Front. Vet. Sci. 6:74. doi: 10.3389/fvets.2019.00074 (“Davis et al.”). There has recently been a reclassification, grouping horses with mild lower airway inflammation, previously termed Inflammatory Airway Disease (IAD), and those with marked lower airway inflammation, previously known as Recurrent Airway Obstruction (RAO), into a unified syndrome, Equine Asthma Syndrome (EAS). See Couetil et al. Equine Asthma Syndrome represents this cohort of horses with lower airway inflammation that exhibit varying clinical presentations. See id.

Treatment of asthmatic horses is multifactorial. The mainstay of therapy varies based on severity and includes inhaled or systemic corticosteroids and bronchodilators (beta adrenergic agonists). Another vital part of management of EAS includes reduced exposure to respirable particles encountered in the normal stable environments, including dust, mold, and bacterial components, that typically trigger airway inflammation and clinical signs. See Couetil et al. ; Davis et al.; Thomas, Sarah J., et al. “Case-Control Study of Risk Factors for Equine Asthma in Texas.” Journal of Equine Veterinary Science, vol. 103, Aug. 2021, https://doi.org/10.1016/j.jevs.2021.103644 (“Thomas et al.”). This environmental modification, while vital, can be difficult to achieve and is potentially the greatest source of client non-compliance. See Couetil et al.

Recent work in people has highlighted potential benefits of dietary modifications as an adjunctive management strategy for human asthma, with a focus on incorporating fruits, vegetables, whole grains, polyunsaturated fats, and vitamin D into the typical Western diet. See Alwarith J, Kahleova H, Crosby L, Brooks A, Brandon L, Levin SM, Barnard ND. The role of nutrition in asthma prevention and treatment. Nutr Rev. 2020 Nov. 1; 78(11):928-938. doi: 10.1093/nutrit/nuaa005. PMID: 32167552; PMCID: PMC7550896 (“Alwarith et al.”). These dietary changes are thought to exert their effects through antioxidant and anti-inflammatory mechanisms that temper airway inflammation and decrease responses to environmental triggers. See id. While altering the diet of asthmatic horses is not a new concept, as practices like soaking hay and feeding pelleted diets are often recommended to reduce exposure to dust, there's a growing recognition that modifying feed components could also offer safer and cost-effective approach in management of EAS to reduce the reliance on corticosteroid therapy. See Couetil et al.

One dietary supplement, resveratrol, has shown promise in improving clinical outcomes in human patients with various diseases, including respiratory conditions, as demonstrated in numerous preclinical and clinical studies. See Martin LM, Johnson PJ, Amorim JR, DeClue AE. Effects of Orally Administered Resveratrol on TNF, IL-1β, Leukocyte Phagocytic Activity and Oxidative Burst Function in Horses: A Prospective, Randomized, Double-Blinded, Placebo-Controlled Study. Int J Mol Sci. 2020 Feb. 20; 21(4):1453. doi: 10.3390/ijms21041453. PMID: 32093379; PMCID: PMC7073105 (“Martin et al.”). Resveratrol's potent anti-inflammatory actions are attributed to several mechanisms, including the reduction of inflammatory cytokine production, suppression of oxidative stress, and modulation of the activity of inflammatory transcription factors. See id. While resveratrol's anti-inflammatory effects have been extensively studied in humans, its impact on inflammation in horses remains largely unexplored. However, a resveratrol-derived supplement has been shown to influence insulin responses in horses with insulin dysregulation. See Manfredi JM, Stapley ED, Nadeau JA, Nash D. Investigation of the Effects of a Dietary Supplement on Insulin and Adipokine Concentrations in Equine Metabolic Syndrome/Insulin Dysregulation. J Equine Vet Sci. 2020 May; 88:102930. doi: 10.1016/j.jevs.2020.102930. Epub 2020 Jan. 22. PMID: 32303322 (“Manfredi et al.”). Further investigation into the anti-inflammatory properties of resveratrol in horses could provide valuable insights into the therapeutic potential of resveratrol-containing products in equine inflammatory diseases, such as EAS.

This study assesses the pulmonary and systemic anti-inflammatory effects of a feed supplement containing resveratrol and other polyphenol antioxidants in healthy and asthmatic horses. It is hypothesized that this respiratory supplement formulation decreases systemic and pulmonary inflammation in both healthy and asthmatic horses and improves bronchoalveolar cytology (BAL) cytology and clinical respiratory signs in asthmatic horses.

### Materials and Methods

There are two parts to this study. Part 1 is performed in healthy and asthmatic horses to ensure the tolerability of the product and assess changes in BAL cytology and systemic and pulmonary inflammatory cytokine concentrations in both groups of horses before and after receiving the supplement. In Part 2, the dietary supplement is administered to a group of previously diagnosed asthmatic horses before induction of lower airway inflammation with hay-feeding. In part 1, supplement-treated horses and an untreated control group on the same farm are evaluated before and after induction of airway inflammation with dusty hay feeding to investigate effects on clinical signs of respiratory disease, clinicopathologic parameters, and BAL cytology and inflammatory cytokine concentrations. Study design was approved by the University of Georgia Institutional Animal Care and Use Committee. Detailed study designs for parts 1 and 2 are discussed separately below.

### Part 1 Study Design: Product Tolerability and Immune Effects in Healthy and Asthmatic Horses

Eighteen university-owned adult horses (twelve Quarter Horses, three Thoroughbreds, and three Warmbloods; eight mares, eight geldings, and two stallions; average age 13.9±4.9 years) are used in Part 1. These horses are categorized into healthy (n=8) or asthmatic (n=10) groups based on a baseline cytology of BAL fluid performed prior to the study. Horses exhibiting more than 5% neutrophils in their BAL cytology are categorized as asthmatic. The threshold of >5% neutrophils in BAL cytology has been previously proposed in the literature and is considered a rather stringent cut-off for a diagnosis of EAS. See Couetil et al.; Rossi, Heini et al. “Comparison of Tracheal Wash and Bronchoalveolar Lavage Cytology in 154 Horses With and Without Respiratory Signs in a Referral Hospital Over 2009-2015.” Frontiers in veterinary science vol. 561. 26 Mar. 2018, doi:10.3389/fvets.2018.00061 (“Rossi et al.”). Of these horses categorized as asthmatic based on BAL % neutrophilia, five are asymptomatic and five show clinical signs such as cough, nasal discharge, or exercise intolerance. The horses are housed on one of two university-owned farms located 3.5 miles (5.6 km) apart in the southeast, with thirteen animals located on one farm and five animals located at the other farm. All horses are housed outside on mixed grass (annual rye, fescue, bermudagrass) pasture for the duration of the study and receive supplemental complete pelleted feed once or twice daily, with some horses also receiving supplemental dry grass hay as needed to maintain their weight. Four of these horses are actively in work, and the other fourteen are not enrolled in an exercise program.

Animals in both healthy and asthmatic groups receive one ounce of a proprietary powdered supplement once daily on top of their morning feed for 6-8 weeks (the exact duration of supplement feeding was determined by animal availability for the post-treatment sampling). Horses in the asthmatic group are managed with attempted reduction in exposure to environmental allergens (housing on pasture, avoidance of the barn during cleaning/bedding) but none are receiving inhaled or systemic corticosteroids or antihistamines for at least four weeks prior to enrollment. Animals in both groups do not receive other supplements or medications during the study period. Part 1 sample collection occurs in the spring and summer over a 2 year period in northeast Georgia, USA.

Group size is determined by a priori sample size calculations performed using an online calculator (http://www.biomath.info/power/ttest.htm) based on previously published data on inflammatory markers in horses with asthma. See Bullone, M., de Lagarde, M., Vargas, A. and Lavoie, J.-.-P. (2015), Serum Surfactant Protein D and Haptoglobin as Potential Biomarkers for Inflammatory Airway Disease in Horses. J Vet Intern Med, 29:1707-1711. https://doi.org/10.1111/jvim.13602 (“Bullone et al.”). Eight animals per group allows detecting an 11-17% difference in inflammatory parameters before and after treatment as significantly different (power 80%, alpha 0.05).

Immediately prior to starting the supplement and after 6-8 weeks of supplement treatment, each horse undergoes a complete physical examination, rebreathing examination by a single investigator (KMA), blood collection, and BAL. Thirty milliliters of blood is collected into clot, lithium heparin, and EDTA tubes, centrifuged at 400×g for 10 minutes, and serum/plasma collected and stored at −80° C. until batch analysis for inflammatory cytokine concentrations via ELISA (see below). Rebreathing examination findings are considered abnormal if any one or more of the following were noted: focal or diffuse adventitious lung sounds, tracheal rattles, cough, or delayed recovery (define as taking more than 5 breaths to return to their baseline respiratory rate and effort).

For BAL fluid collection, horses are sedated with xylazine (0.5 mg/kg IV) and butorphanol (0.05 mg/kg IV). A 10 mm diameter, 2.4 m BAL catheter (Jorgenson laboratories, Loveland, CO) is passed via nasal approach until wedged into a bronchus. The lavage solution consists of 4 aliquots of 50 mL physiologic saline (0.9% NaCl) solution infused and aspirated immediately with the recovered volume recorded.

After determination of BAL fluid nucleated cell count, bronchoalveolar fluid is centrifuged at 200×g for 10 minutes and supernatant BAL fluid is aliquoted and frozen at −80° C. until batch analysis for inflammatory cytokine concentrations via ELISA (see below).

### Part 2 Study Design: Asthma Induction

In Part 2, the dietary supplement is tested on previously diagnosed asthmatic horses with induced lower airway inflammation to identify any effects on BAL cytology and inflammatory cytokine concentrations, in addition to clinical signs and routine hematology and biochemistry. Ten university-owned adult horses (eight Quarter Horses, one Warmblood, and one Arabian; four geldings and six mares; average age 19.8 ±4.5 years) are used in Part 2, with the same inclusion criteria for EAS employed as for animals in Part 1. Horses are randomized into two treatment groups using an online randomizer (https://www. graphpad.com/quickcalcs/randomize1/) with five horses randomized into a treatment group to receive one ounce of the same proprietary powdered supplement once daily on top of their morning feed for eight weeks, and five animals comprising an untreated control group. The treatment and control groups are housed on the same farm in adjacent pastures for the duration of this part of the study. The study is conducted in the late fall and early winter in northeast Georgia when environmental allergens are generally lower and pasture is relatively dormant.

For the first four weeks of treatment, all animals are managed with a low dust diet (pelleted feed and moistened square bale hay) to decrease as much airway inflammation as possible. After this acclimation period, all ten horses are switched from wet square bale hay to dry, dusty round bales of bermudagrass hay from the same cutting of hay. Exposure to dusty hay is a well-described model used to induce airway inflammation in asthmatic horses. See Westerfeld R, Payette F, Dubuc V, et al. Effects of soaked hay on lung function and airway inflammation in horses with severe asthma. J Vet Intern Med. 2024; 38(1): 469-476. doi: 10.1111/jvim.16919 (“Westerfeld et al.”). Again, horses do not receive other supplements or medications during the treatment period. A rescue protocol is set in place: any horse showing severe clinical signs (i.e., persistent cough, increased respiratory effort at rest, respiratory distress) is treated with the Aservo Inhaler, an FDA approved treatment for EAS containing the corticosteroid ciclesonide.

Group size for Part 2 is also determined by a priori sample size calculations performed using an online calculator (http://www.biomath.info/power/ttest.htm) based on changes in BAL cytology in asthmatic horses detected in Part 1 of the study (reduction in BAL % neutrophils, from 12.64±9.34% to 4.36±1.16%). This reveals that <6 animals/group allows detecting a similar 2.9-fold difference in BAL neutrophilia between groups (power 80%, alpha 0.05).

Prior to initiation of the supplement in the treatment group and the first four week low-dust diet period, all horses have 30 ml of whole blood collected from the jugular vein by direct venipuncture for routine hematology (Siemens Advia 2120i, St. Paul, MN) and plasma biochemistry (Roche Cobas 6000/c501, Indianapolis, IN) analysis. After the first four week low-dust diet period, each horse undergoes physical examination, rebreathing examination by the same investigator (KMA), BAL, and blood collection again to assess baseline clinical and inflammatory parameters prior to the four-week airway inflammation-induction period. These procedures, and routine hematology and biochemistry blood panels, are repeated after the four-week airway inflammation-induction period to assess the effects of the supplement on induced airway inflammation and routine hematology/biochemistry findings. The BAL procedure, cytologic analysis, and serum and BAL processing and storage are performed as for Part 1 of the study. The investigators performing the rebreathing exam (KMA) and the BAL cytology (LJB) analysis are blinded to the animals' treatment group assignments.

### Assessment of Serum and Pulmonary Immune and Inflammatory Markers in Part 1 and 2

Both serum and BAL fluid are assayed for the following inflammatory and immune markers that have been demonstrated in previous studies to be associated with the incidence or severity of asthma in horses: interleukin (IL)-17, IL-10, IL-4, IL-1β, tumor necrosis factor (TNF)-α, and IL-6. See Simões J, Batista M, Tilley P. The Immune Mechanisms of Severe Equine Asthma-Current Understanding and What Is Missing. Animals (Basel). 2022 Mar. 16; 12(6):744. doi: 10.3390/ani12060744. PMID: 35327141; PMCID: PMC8944511 (“Simoes et al.”); Couetil, Laurent, et al. “Equine Asthma: Current Understanding and Future Directions.” Frontiers in Veterinary Science, vol. 7, 2020, https://doi.org/10.3389/fvets.2020.00450 (“Couetil2 et al.”). A previously validated bead-based multiplex immunoassay is used to quantify IL-17, IL-10, and IL-4 in serum and BAL fluid as previously described. See Wagner B, Freer H. Development of a bead-based multiplex assay for simultaneous quantification of cytokines in horses. Vet Immunol Immunopathol. 2009 Feb. 15; 127(3-4): 242-8. doi: 10.1016/j.vetimm.2008.10.313. Epub 2008 Oct. 18. PMID: 19027964 (“Wagner et al.”); Bordin, A.I., Cohen, N.D., Giguère, S. et al. Host-directed therapy in foals can enhance functional innate immunity and reduce severity of Rhodococcus equipneumonia. Sci Rep 11, 2483 (2021). https://doi.org/10.1038/s41598-021-82049-y (“Bordin et al.”). Serum and BAL fluid IL-1β, IL-6, and TNF-α are quantified via a commercial ELISA (R&D Duoset, Minneapolis, MN) as per the manufacturer instructions.

### Analysis and Statistics

Data distribution is assessed with the Shapiro-Wilk test. Since group sizes are small and the majority of the data is not normally distributed, non-parametric analyses are used for two-way comparisons. In Part 1, comparisons between healthy and asthmatic groups before and after treatment are conducted with linear mixed effects analysis with disease category (healthy or asthma) and time and time x disease as fixed effects and horse as a random effect. In Part 2, comparisons between supplement-treated and control horses are conducted with linear mixed effects analysis with time and treatment and time x treatment as fixed effects and horse as a random effect. Post-hoc pair-wise comparisons are performed using the method of Sîdak. The change in parameters over time (delta) is calculated for some variables by subtracting baseline values from post-treatment values and compared within and between groups with Wilcoxon rank sum tests and Mann Whitney U tests. Chi square tests with Yates correction are used to compare the proportion of horses with abnormal rebreathing exams between the healthy and control groups in Part 1, and between the treatment and control groups in Part 2. Analyses are conducted using commercial statistical software (GraphPad Prism v. 10.0, GraphPad Software LLC, Boston, MA, USA) and p <0.05 is considered statistically significant for all analyses.

### Results

### Part 1: Product Tolerability and Clinical Effects in Healthy and Asthmatic Horses

The dietary supplement is palatable, and no apparent adverse effects due to the supplement are noted during the 6-8 week feeding period in the healthy or asthmatic groups.

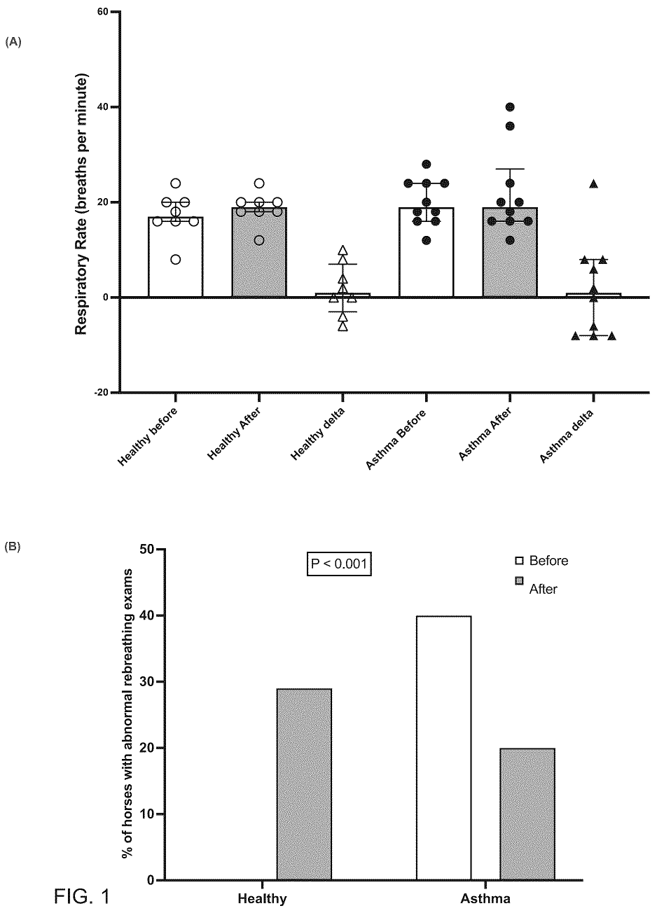

Respiratory rates and the proportion of horses with abnormal rebreathing exams during the study period are shown in FIG. 1, which illustrates respiratory rates (A) and the proportion of horses with abnormal rebreathing exam (B) before and after 6-8 weeks of mixed polyphenol supplementation in eight healthy horses (open circles) and ten horses previously diagnosed with mild equine asthma (closed circles). The change in respiratory rate over the treatment period (after treatment—before treatment) is shown in (A) with open triangles in healthy horses and closed triangles in asthmatic horses. There were no significant differences in respiratory rate (A) between or within groups over time (P>0.302). There was a significant difference in the proportion of horses in the asthmatic group with an abnormal rebreathing exam (B) before and after supplement treatment compared to the healthy group (P<0.001, 95% confidence interval (CI)=0.55 to 0.87), with an increase over the treatment period in the healthy horses and a decrease during that period in the asthmatic horses.

### Effects on BAL Fluid Cytology—Part 1

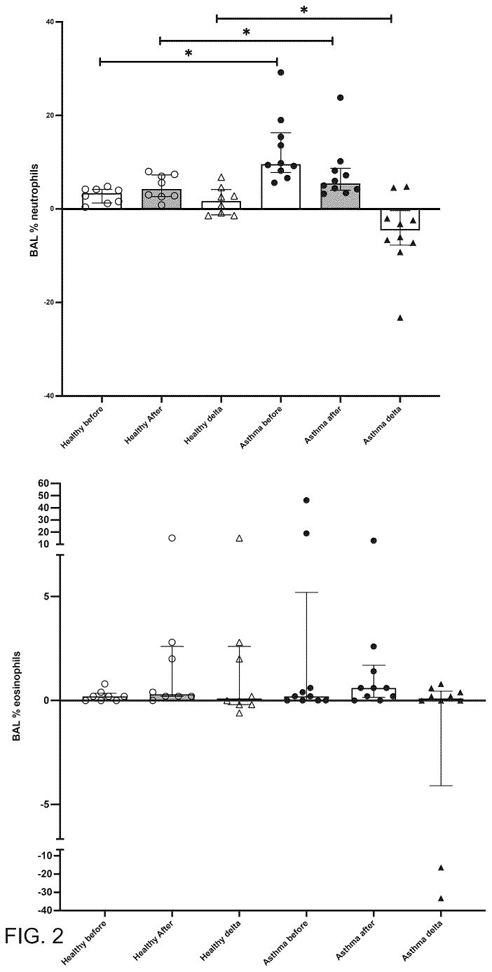

FIG. 2 illustrates the BAL % neutrophils and delta BAL % neutrophils (after treatment—before treatment) in the healthy and asthmatic groups before and after 6-8 weeks of supplement treatment in Part 1 of the study. There is an overall significant effect of health status (healthy vs. asthma, P=0.006; 95% CI −10.49 to −2.13 ) and a significant interaction between health status and time (P=0.036; 95% CI −13.09 to −0.49 ), with significantly higher BAL % neutrophils in the asthmatic horses than the healthy group before treatment (P<0.001; 95% CI=−14.73 to −4.67 %). Additionally, within the asthmatic group, there is a significant decrease in BAL % neutrophils after the supplement treatment as compared to the baseline measurement (P=0.022; 95% CI 0.84 to 9.23 %). There is also a significantly larger change in BAL % neutrophils (delta BAL % neutrophils) in asthmatic horses during the supplement treatment period as compared to the healthy horses (p=0.017).

There is no effect of disease status (healthy or asthmatic) or of supplement treatment or interaction between disease and treatment on BAL % eosinophils or delta BAL % eosinophils in either group (FIG. 2; P≥0.12). All healthy horses have a BAL % eosinophils less than published guidelines for EAS diagnosis at the start of the study (>3%; Couëtil, 2016), and these remain low in all but one horse (15.2%) at the after treatment timepoint. There are 2 horses in the asthmatic group with an increased BAL % eosinophils (46.2% and 19.0% respectively) prior to supplement treatment. These percentages do decrease after treatment to 13% and 2.6% respectively, but a significant effect of treatment on BAL % eosinophils or delta BAL % neutrophils is not found (P≥0.421).

### Serum and BAL Cytokine Analysis Part 1

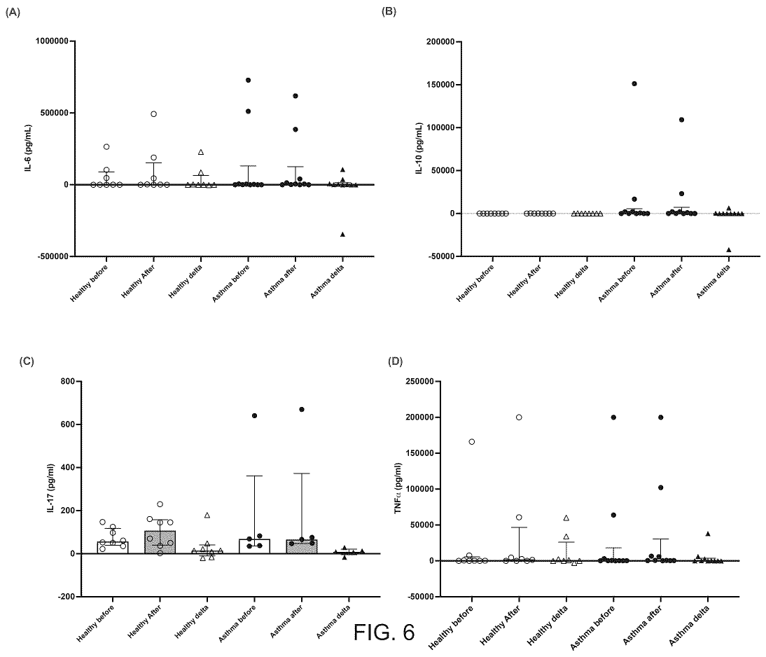

Serum and BAL concentrations of IL-1β and IL-4 both groups and at both time points are below the limit of detection of the assays used in the majority of animals, so further analysis is not performed for these cytokines (data not shown). Serum IL-6, IL-10, TNF-α, and IL-17 concentrations in healthy and asthmatic horses before and after supplement treatment in Part 1 of the study are shown in FIG. 6. There are no significant differences (P<0.06) between or within groups. There is no significant effect of disease status, supplement treatment, or significant disease x treatment interactions on serum concentrations of any cytokine (P=0.063-0.962).

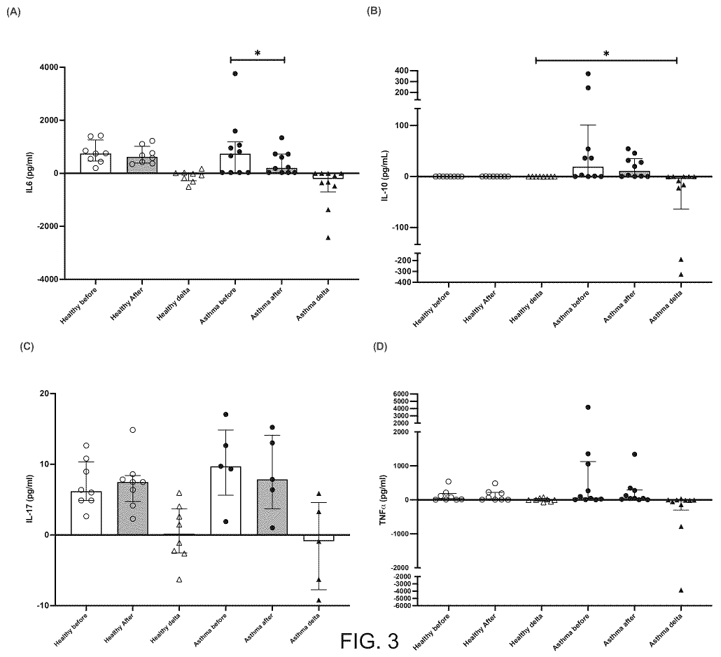

BAL cytokine concentrations are shown in FIG. 3. There is no significant overall effect of disease status, supplement treatment, or disease x treatment interaction on IL-10, IL-17 or TNF-α concentrations in BAL fluid (P=0.093−0.68). There is an almost significant overall effect of supplement treatment on BAL IL-6 concentrations (P=0.051; 95% CI=−1.04 to 613.5 pg/ml), so post-hoc testing is performed to investigate this further. This reveals that BAL fluid IL-6 concentrations are similar before and after supplement treatment in healthy horses (P=0.62), while asthmatic horses have significantly lower BAL fluid IL-6 concentration after supplement treatment compared to before treatment (P=0.02; 95% CI=93.86 to 913.2 pg/ml). There is also a significantly greater decrease (delta) in BAL fluid IL-10 concentrations in asthmatic horses compared to healthy horses over the course of the study (P=0.02).

### Part 2: Asthma Induction Study

### Tolerability and Effects on Clinical Respiratory Status—Part 2

In Part 2 of the study, all horses in the treatment group again readily consume the dietary supplement, and no apparent adverse clinical or clinicopathologic effects are noted in either group during the 8-week feeding period.

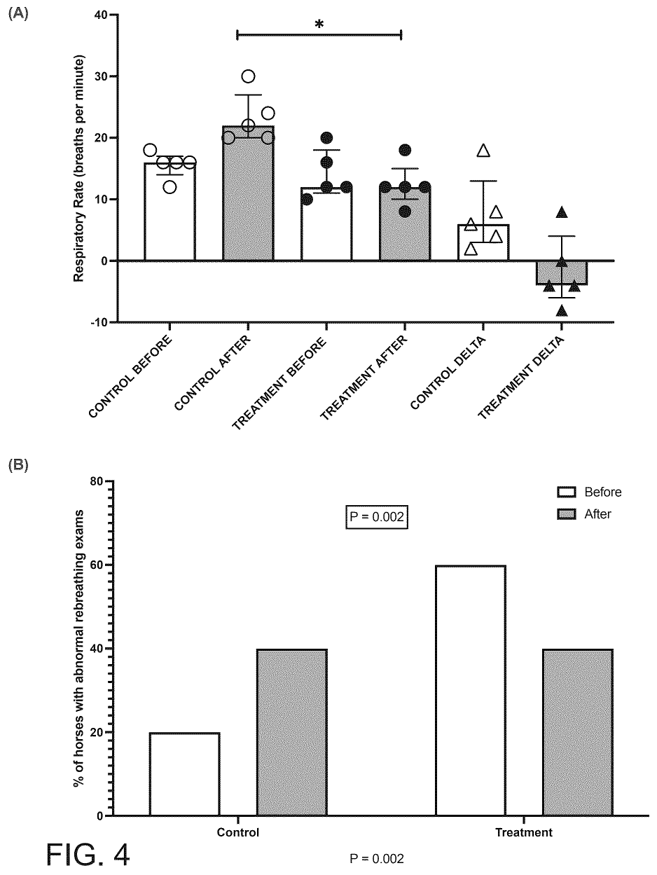

Respiratory rates and the proportion of horses with abnormal rebreathing exams during the treatment/asthma induction period are shown in FIG. 4. There is a significant effect of treatment (P=0.001; 95% CI 2.8 to 9.6 breaths per minute) and treatment x time interaction (P=0.01; 95% CI=2.4 to 16.0) on respiratory rate. Due to the significant interaction effect, two-way group comparisons are performed to further investigate the effect of time and treatment in the control and treatment group. Respiratory rate is similar in the control and treatment at the start of the study (P=0.52) but is significantly lower in treated asthmatic horses compared to control asthmatic horses after the supplement treatment period (P=0.008). The delta respiratory rate over the treatment/asthma induction period in treated horses is a median decrease of 4 breaths per minute compared to control horses with a median increase of 6 breaths per minute, but this does not differ significantly between groups (P=0.07).

There is a significant difference in the proportion of horses in the treatment group with an abnormal rebreathing exam (B) before and after asthma induction compared to the control group (P=0.002).

There is also a significant difference (P=0.002; 95% CI=0.11 to 0.41) between treatment and control groups in the proportion of horses with an abnormal rebreathing exam over the course of the treatment period. In the control group, 20% of horses (1/5) have an abnormal rebreathing exam at the start of the study and 40% (2/5) have an abnormal rebreathing exam at the end of the asthma induction period. In contrast, in the treatment group, 60% (3/5) of the horses have an abnormal rebreathing exam at the start of the treatment period, compared to 40% (2/5) at the end of the asthma induction period.

### BAL Fluid Cytology—Part 2

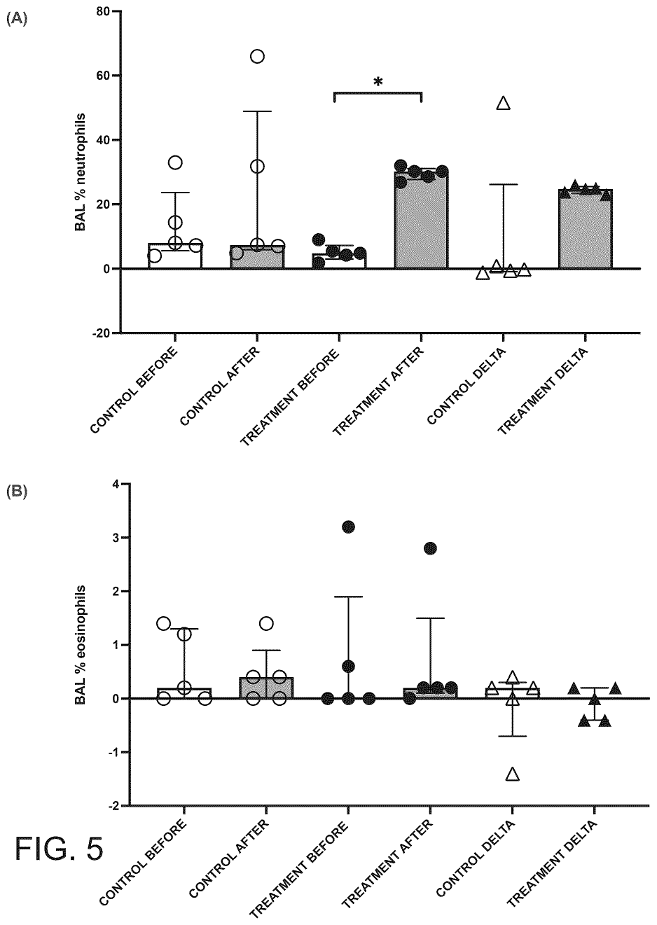

The percent of neutrophils and eosinophils in the BAL fluid in both groups before and after the asthma induction period and the delta BAL % neutrophils and eosinophils over time are shown in FIG. 5. There is a significant overall effect of time (P=0.01; 95% CI=−29.29 to −5.31) on BAL % neutrophils with a significant increase in BAL % neutrophils before and after asthma induction in the treatment group (P=0.01; 95% CI=−41.47 to −7.57 ). There is no significant effect of treatment or treatment x time interaction (P≥0.20) on BAL % neutrophils, and no significant effect of treatment, time or treatment x time interaction (P≥0.59) on BAL % eosinophils. Delta % neutrophils or eosinophils also does not differ significantly between groups (P≥0.15).

### BAL Fluid Cytokine Analysis Part 2

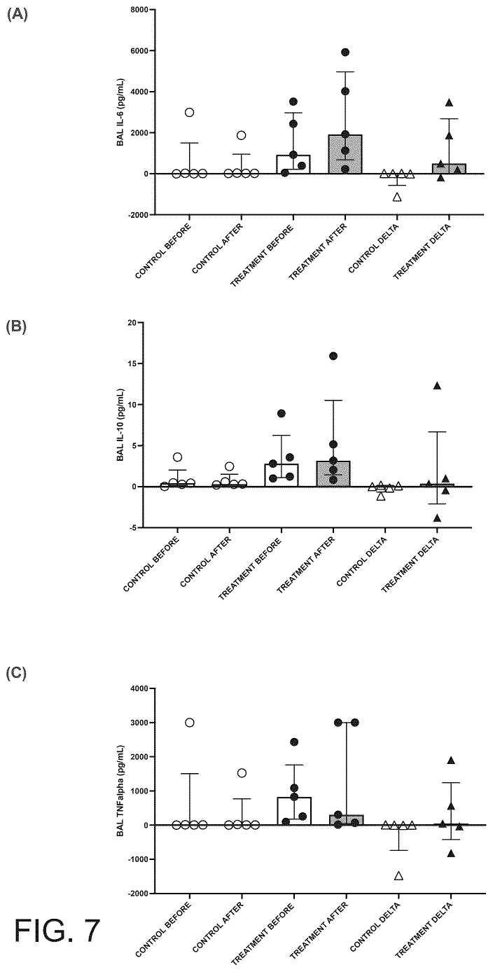

Based on findings in part 1, only BAL fluid concentrations of IL-6, IL-10 and TNF-α are assessed in Part 2 and are shown in FIG. 7. There are no significant differences (P≤0.08) between or within groups. There is no significant overall effect of time or treatment or treatment x time interaction on BAL fluid IL-6. IL-10, or TNF-α IL-6 concentrations (P≥0.08). Delta cytokine concentrations also do not differ significantly between the treatment and control groups (P≥0.10).

### Discussion

The results of this study support the hypothesis that the mixed polyphenol antioxidant supplement modulates pulmonary inflammation in horses with EAS, demonstrating improvement in BAL neutrophilia and pulmonary pro-inflammatory cytokine production across the two parts of the study. In addition, there appears to be some positive effect on clinical respiratory signs in horses with both naturally-occurring and induced airway inflammation in Parts 1 and 2, respectively. The supplement is consumed readily as a top-dress on feed by all horses and appeared well tolerated. No adverse clinical side effects or clinicopathologic changes are noted throughout the study. The supplement does not appear to induce pulmonary inflammation in normal or asthmatic horses.

Although there is no significant effect on respiratory rate in Part 1, there is a significant decrease in the percentage of asthmatic horses with an abnormal rebreathing exam compared to healthy horses. The challenge with assessing clinical signs in this population of horses is that many are not in work at the time of the study, and none had severe asthma with abnormal respiratory status at rest. In addition, based on pre-treatment BAL neutrophilia, most of the asthmatic group would have been classified as mild/moderate asthmatics, with BAL fluid neutrophilia between 5-20%. See Davis et al. Mild/moderate asthma in horses can be subclinical at rest, with other clinical signs including prolonged recovery after exercise or poor performance. See Couetil2 et al. Given that few of the horses in this study were in work during the study period, it is difficult to determine if the supplement influences other clinical signs associated with mild EAS.

There is a significant change in BAL fluid neutrophil percentage between healthy and asthmatic horses identified in Part 1, with improved BAL fluid neutrophilia in asthmatic horses after 6-8 weeks of supplement treatment compared to the start of the study. This improvement in BAL cytology is accompanied by a decrease in concentrations of the inflammatory cytokines IL-6 and IL-10 in the BAL fluid in asthmatic horses. IL-6 is a multifunctional cytokine that has multiple effects in inflammation and disease, and increased pulmonary concentrations have been associated with the incidence and severity of equine asthma. See Simoes et al; Couetil2 et al. Produced by various cells including immune cells, fibroblasts, and endothelial cells, IL-6 plays a pivotal role in the immune response and inflammation regulation. It induces the synthesis of acute phase proteins, stimulates antibody production and effector T cell development, and stimulates proliferation of several non-immune cells. Beyond its immunological functions, IL-6 has been implicated in various diseases, including autoimmune disorders, cardiovascular diseases, and cancer, making it a significant target for therapeutic intervention and research. See Tanaka T, Narazaki M, Kishimoto T. IL-6 in inflammation, immunity, and disease. Cold Spring Harb Perspect Biol. 2014 Sep. 4; 6(10):a016295. doi: 10.1101/cshperspect.a016295. PMID: 25190079; PMCID: PMC4176007 (“Tanaka et al.”).

In Part 2, both a control group of untreated asthmatic horses and a treatment group receiving the supplement are included and airway inflammation is induced in both groups by 30 days of exposure to dusty hay (REF). There continues to be an improvement in clinical respiratory signs in horses receiving the supplement, with significant improvement in respiratory rate in treated horses compared to control horses after the treatment period. In addition, consistent with the result in Part 1, there is a significantly lower proportion of horses with rebreathing exam abnormalities in the treatment group compared to the control group over the study course.

Of particular note, the clinical improvement in respiratory signs in Part 2 occurs in the presence of apparently increasing airway inflammation with asthma induction. A significant increase in BAL neutrophilia is observed over the 30-day dusty hay exposure period, as was expected. All (5/5) horses in the treatment group, but only 2/5 horses in the control group demonstrate BAL % neutrophils >20%, consistent with a diagnosis of severe equine asthma (see Davis et al.), after dusty hay exposure. This variability in airway neutrophilic response to dusty hay exposure suggests that some of the horses randomly assigned to the control group may have had milder disease than horses randomized to the treatment group, and makes between group comparisons related to airway inflammatory parameters hard to interpret.

Although a potential anti-inflammatory effect of the supplement with regards to BAL cytokine production is observed in Part 1, a significant effect of supplement treatment on BAL pro-inflammatory cytokine production is not observed in Part 2 after airway inflammation was induced with dusty hay. In addition, substantial variability is observed in the BAL cytokines measured among individual animals. As mentioned above, the treated asthmatic horse in Part 2 may have been more severely affected than the control group based on clinical signs and BAL fluid cytology, complicating interpretation Part 2.

Advantageously, the supplement can be incorporated with feed components in the normal diet of an asthmatic horse. The addition the polyphenol-based supplement, which can contain resveratrol and other polyphenol antioxidants, may be a safe and cost-effective adjunct option for treating asthmatic horses.

This study had some limitations. The design of Part 1 enables the assessment of the temporal effects of 6-8 weeks of supplement treatment in healthy and asthmatic animals compared to their baseline parameters, but does enable determining the relative impact of changes in environmental allergens during this period. Thus, it is possible that the improvement in rebreathing exam, airway neutrophil, and BAL cytokine concentrations that is observed in asthmatic horses receiving the supplement in Part 1 could at least in part reflect improvement in airway inflammation associated with changing environmental allergens during the study period. However, given that this study is conducted in the spring/summer in the southeast - a time in which environmental-associated allergens are generally high—and involves horses on two different farms over two different years, it seems less likely that this is purely an effect of changing environment. Additionally, in this study, horses are classified as healthy or asthmatic solely based on BAL fluid cytology, which can vary in individual animals over time and does not always correlate with abnormalities in lung function, airway hyperreactivity, and other clinical signs in horses with equine asthma. See Davis et al. Lastly, while the study population numbers in both parts were determined by a priori sample size calculations, sample sizes in all groups in both Parts 1 and 2 are small and there is substantial inter-individual variation in some parameters measured (e.g. BAL cytology and BAL cytokine concentrations). Further investigation may determine if these results are consistent in a larger population of horses with naturally occurring equine asthma.

In sum, the results of this study suggest that a resveratrol/polyphenol anti-oxidant-based feed supplement may reduce some markers of airway inflammation and improve some clinical signs in mild-to-moderately affected asthmatic horses that are undergoing concurrent environmental allergen management including outdoor housing and dietary dust/hay avoidance. Substantial anti-inflammatory effects of supplement administration is not observed in more severely affected asthmatic horses with airway inflammation induced by dusty hay exposure in the study, but despite this, these horses show significant improvement in clinical signs compared to untreated, challenged horses.

The polyphenol-based supplement may have benefit as an adjunctive management strategy in horses with mild to moderate disease or in animals with severe disease in conjunction with other effective therapies such as corticosteroids. Larger scale study in a clinical population of asthmatic horses may determine if the mixed polyphenol supplement can effectively reduce airway inflammation/reactivity in susceptible horses to a degree that permits reduced corticosteroid use. This may limit potential steroid-related side effects, and lessen the financial strain of prolonged treatment and the challenges of maintaining a dust-free environment that many horse owners struggle with. See Couetil2 et al.

All documents described herein are incorporated by reference herein, including any priority documents and or testing procedures to the extent they are not inconsistent with this text. The following documents are incorporated by reference in their entirety:
Couëtil, L.L., et al. “Inflammatory Airway Disease of Horses—Revised Consensus Statement.” Journal of Veterinary Internal Medicine, vol. 30, no. 2, 2016, pp. 503-515., https://doi.org/10.1111/jvim.13824.
Davis KU and Sheats MK (2019) Bronchoalveolar Lavage Cytology Characteristics and Seasonal Changes in a Herd of Pastured Teaching Horses. Front. Vet. Sci. 6:74. doi: 10.3389/fvets.2019.00074
Thomas, Sarah J., et al. “Case-Control Study of Risk Factors for Equine Asthma in Texas.” Journal of Equine Veterinary Science, vol. 103, August 2021, https://doi.org/10.1016/j.jevs.2021.103644.
Alwarith J, Kahleova H, Crosby L, Brooks A, Brandon L, Levin SM, Barnard ND. The role of nutrition in asthma prevention and treatment. Nutr Rev. 2020 Nov. 1; 78(11):928-938. doi: 10.1093/nutrit/nuaa005. PMID: 32167552; PMCID: PMC7550896.
Martin LM, Johnson PJ, Amorim JR, DeClue AE. Effects of Orally Administered Resveratrol on TNF, IL-1β, Leukocyte Phagocytic Activity and Oxidative Burst Function in Horses: A Prospective, Randomized, Double-Blinded, Placebo-Controlled Study. Int J Mol Sci. 2020 Feb. 20; 21(4): 1453. doi: 10.3390/ijms 21041453. PMID: 32093379; PMCID: PMC7073105.
Manfredi JM, Stapley ED, Nadeau JA, Nash D. Investigation of the Effects of a Dietary Supplement on Insulin and Adipokine Concentrations in Equine Metabolic Syndrome/Insulin Dysregulation. J Equine Vet Sci. 2020 May; 88:102930. doi: 10.1016/j.jevs.2020.102930. Epub 2020 Jan. 22. PMID: 32303322.
Rossi, Heini et al. “Comparison of Tracheal Wash and Bronchoalveolar Lavage Cytology in 154 Horses With and Without Respiratory Signs in a Referral Hospital Over 2009-2015.” Frontiers in veterinary science vol. 5 61. 26 Mar. 2018, doi: 10.3389/fvets.2018.00061
Bullone, M., de Lagarde, M., Vargas, A. and Lavoie, J.-.-P. (2015), Serum Surfactant Protein D and Haptoglobin as Potential Biomarkers for Inflammatory Airway Disease in Horses. J Vet Intern Med, 29: 1707-1711. https://doi.org/ 10.1111/jvim.13602
Westerfeld R, Payette F, Dubuc V, et al. Effects of soaked hay on lung function and airway inflammation in horses with severe asthma. J Vet Intern Med. 2024; 38(1): 469-476. doi: 10.1111/jvim.16919
Simões J, Batista M, Tilley P. The Immune Mechanisms of Severe Equine Asthma-Current Understanding and What Is Missing. Animals (Basel). 2022 Mar. 16; 12(6):744. doi: 10.3390/ani12060744. Pmid: 35327141; Pmcid: Pmc8944511.
Couetil, Laurent, et al. “Equine Asthma: Current Understanding and Future Directions.” Frontiers in Veterinary Science, vol. 7, 2020, https://doi.org/10.3389/fvets.2020.00450.

Wagner B, Freer H. Development of a bead-based multiplex assay for simultaneous quantification of cytokines in horses. Vet Immunol Immunopathol. 2009 Feb. 15; 127(3-4):242-8. doi: 10.1016/j.vetimm.2008.10.313. Epub 2008 Oct. 18. PMID: 19027964.
Bordin, A.I., Cohen, N.D., Giguère, S. et al. Host-directed therapy in foals can enhance functional innate immunity and reduce severity of Rhodococcus equi pneumonia. Sci Rep 11, 2483 (2021). https://doi.org/10.1038/s41598-021-82049-y.
Tanaka T, Narazaki M, Kishimoto T. IL-6 in inflammation, immunity, and disease. Cold Spring Harb Perspect Biol. 2014 Sep. 4; 6(10):a 016295. doi: 10.1101/cshperspect.a016295. PMID: 25190079; PMCID: PMC4176007.

As is apparent from the foregoing general description and the specific embodiments, while forms of the present disclosure have been illustrated and described, various modifications can be made without departing from the spirit and scope of the present disclosure. Accordingly, it is not intended that the present disclosure be limited thereby.

The specific embodiments described herein have been illustrated by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for (perform)ing (a function) . . . ” or “step for (perform)ing (a function) . . . ”, it is intended that such elements are to be interpreted under 35 U.S.C. 112(f). However, for any claims containing elements designated in any other manner, it is intended that such elements are not to be interpreted under 35 U.S.C. 112(f).

While the present disclosure has been described with respect to a number of embodiments and examples, those skilled in the art, having benefit of this disclosure, will appreciate that other embodiments can be devised which do not depart from the scope and spirit of the present disclosure.

What is claimed is:

1. A composition comprising one or more polyphenols, wherein
the one or more polyphenols are present in a therapeutically effective amount for treating Equine Asthma Syndrome in an equine in need thereof, and
wherein the one or more polyphenols comprise resveratrol and at least one of citrus bioflavonoid and hesperidin.

2. (canceled)

3. The composition of claim 1, wherein the one or more polyphenols comprise:
resveratrol in an amount between 0.5% by weight to 10% by weight of the composition as a whole;
and at least one of:
citrus bioflavonoid in an amount between 5% by weight to 30% by weight of the composition as a whole; and
hesperidin in an amount between 0.5% by weight to 10% by weight of the composition as a whole.

4-6. (canceled)

7. The composition of claim 1, wherein the one or more polyphenols include quercetin.

8. The composition of claim 7, wherein quercetin is present in an amount between 0.1% by weight to 5% by weight, by weight of the composition as a whole.

9. (canceled)

10. The composition of claim 1, further comprising one or more amino acids.

11. The composition of claim 10, wherein the one or more amino acids include at least one of lysine and leucine.

12. (canceled)

13. The composition of claim 10, wherein the one or more amino acids include:
leucine in an amount between 1% by weight to 10% by weight, by weight of the composition as a whole;
lysine in an amount between 1% by weight to 10% by weight, by weight of the composition as a whole; or
a combination thereof.

14. (canceled)

15. The composition of claim 1, further comprising a carrier.

16. The composition of claim 15, wherein the carrier includes active dry yeast.

17. (canceled)

18. The composition of claim 1, further comprising an additive, wherein the additive includes a flavoring agent, a sweetening agent, or a combination thereof.

19. (canceled)

20. A method for treating Equine Asthma Syndrome in an equine in need thereof, the method comprising administering the composition of claim 1 to the equine.

21. A method for treating Equine Asthma Syndrome, the method comprising administering to the equine a composition comprising:
resveratrol in an amount between 0.5% by weight to 10% by weight of the composition as a whole;
and at least one of:
citrus bioflavonoid in an amount between 5% by weight to 30% by weight of the composition as a whole; and
hesperidin in an amount between 0.5% by weight to 10% by weight of the composition as a whole.

22-28. (canceled)

29. A method for treating an airway inflammation disease in an animal in need thereof, the method comprising administering a composition including a therapeutically effective amount of one or more phenols to the animal.

30. The method of claim 29, wherein the composition comprises one or more phenols and at least one of: one or more amino acids, a carrier, and an additive.

31-32. (canceled)

33. The method of claim 29, wherein administering the composition comprises orally administering the composition.

34. (canceled)

35. The method of claim 29, wherein the one or more polyphenols comprise resveratrol and at least one of citrus bioflavonoid and hesperidin.

36. A method for treating airway inflammatory responses to environmental triggers in an animal in need thereof, the method comprising administering the composition of claim 1 to the animal.

37. The method of claim 36, wherein the composition comprises one or more phenols and at least one of: one or more amino acids, a carrier, and an additive.

38-39. (canceled)

40. The method of claim 36, wherein administering the composition comprises orally administering the composition.

41. (canceled)

42. The method of claim 36, wherein the one or more polyphenols comprise resveratrol and at least one of citrus bioflavonoid and hesperidin.
